(19) United States
(12) Patent Application Publication
(10) Pub. No.: US 2012/0046251 A1
(43) Pub. Date: Feb. 23, 2012

(54) INHIBITORS OF SOLUBLE EPOXIDE HYDROLASE TO INHIBIT OR PREVENT NIACIN-INDUCED FLUSHING

(75) Inventors: Saul Schaefer, Davis, CA (US); Bruce D. Hammock, Davis, CA (US); Ahmet Bora Inceoglu, Davis, CA (US)

(73) Assignee: The Regents of the University of California, Oakland, CA (US)

(21) Appl. No.: 13/260,565

(22) PCT Filed: Apr. 5, 2010
(86) PCT No.: PCT/US10/29971

§ 371 Date: Nov. 1, 2011

Related U.S. Application Data
(60) Provisional application No. 61/166,957, filed on Apr. 6, 2009.
(60) Provisional application No. 61/258,271, filed on Nov. 5, 2009.

(51) Int. Cl. A61K 31/60; A61K 31/4406; A61P 17/00; A61K 31/17; A61K 31/215; A61K 31/336; A61K 31/404

(52) U.S. Cl. 514/163; 514/475; 514/356; 514/411; 514/588; 514/529

(57) ABSTRACT

The invention discloses methods of using cis-epoxyeicosantrienoic acids (“EETs”), inhibitors of soluble epoxide hydrolase (“sEH”), or a combination of an EET and an inhibitor of sEH, to reduce or prevent niacin-induced cutaneous vasodilation (“flushing”) in subjects suffering from this undesirable side effect of receiving therapeutic amounts of niacin.

### CROSS-REFERENCE TO RELATED APPLICATIONS

The present application claims the benefit of U.S. Provisional Application No. 61/166,957, filed on Apr. 6, 2009 and U.S. Provisional Application No. 61/258,271, filed on Nov. 5, 2009, the disclosures of each of which are hereby incorporated herein by reference in their entirety for all purposes.

### STATEMENT AS TO RIGHTS TO INVENTIONS MADE UNDER FEDERALLY SPONSORED RESEARCH AND DEVELOPMENT

This invention was made with government support under grant number ES002710, awarded by the National Institutes of Health and by the Veterans Administration. The government has certain rights in this invention.

### FIELD OF THE INVENTION

The present invention relates to the inhibition or prevention of niacin-induced flushing by the administration of inhibitors of soluble epoxide hydrolase, epoxygenated fatty acids or mixtures thereof.

### BACKGROUND OF THE INVENTION

Niacin or nicotinic acid (pyridine-3-carboxylic acid) is an effective agent to reduce cholesterol and the consequences of coronary artery disease, including heart attacks and death. Unfortunately, the use of niacin is severely limited by its major side effect, cutaneous vasodilation or “flushing.” The flushing response can include cutaneous redness, itching and/or tingling. Flushing affects approximately 50% of patients and causes a high rate of discontinuation in a majority of these patients. Flushing may not only cause disabling discomfort and interference with normal activities, but may also result in significant morbidity and mortality by limiting the use of life-saving therapeutic agents such as niacin. An agent which can limit flushing can therefore have substantial clinical value.

Several strategies to reduce flushing have been tried, including the use of high doses of aspirin (e.g., 300 mg or more), but these have limited efficacy. Moreover, the administration of high doses of aspirin over time is undesirable.

Studies have elucidated some of the mechanisms of niacin-induced flushing and, specifically, have determined that stimulation of the DP1 receptor by prostaglandin D2 (PGD2) causes flushing. See, e.g., Cheng, et al., (2006) Proc Natl Acad Sci 103(17):6682-7. Niacin administration or PGD2 administration both lead to flushing. An antagonist of PGD2 receptor (DP1) has been described and was shown to reduce niacin-induced flushing. See, Lai, et al., Clin Pharmacol Ther. (2007) 81(6):849-57. See also, WO 2008/097535; WO 2006/089309 and WO 2004/103370. This compound is now approved in Europe, but was not approved for clinical use in the United States.

There remains a need for efficacious, safe and low-cost agents for the inhibition and/prevention of niacin-induced flushing.

### BRIEF SUMMARY OF THE INVENTION

The present invention provides methods to prevent, reduce or block substantial flushing (e.g., frequency and/or severity) as a side effect during the treatment of humans for atherosclerosis, dyslipidemia, diabetes and related conditions using nicotinic acid or another nicotinic acid receptor agonist.

Accordingly, in one aspect, the invention provides methods of reducing or preventing niacin-induced cutaneous vasodilation in a subject in need thereof, said method comprising administering to said subject an effective amount of an agent or agents selected from the group consisting of (i) an inhibitor of sEH, (ii) an epoxygenated fatty acid, or (iii) both an inhibitor of sEH and an epoxygenated fatty acid, thereby reducing or preventing said niacin-induced cutaneous vasodilation in said subject.

In some embodiments, the epoxygenated fatty acid is a cis-epoxyeicosantrienoic acid (“EET”). In some embodiments, the EET is selected from the group consisting of 14,15-EET, 8,9-EET, 11,12-EET or 5,6-EET. In some embodiments, the EET is synthetic or an EET analog.

In some embodiments, the agent is an inhibitor of sEH. In some embodiments, the inhibitor of sEH has a primary pharmacophore selected from the group consisting of a urea, a carbamate and an amide. In some embodiments, the inhibitor of sEH is selected from the group consisting of t-AUCB, trans-4-[4-(3-adamantan-1-ylureido)cyclohexyloxy]benzoic acid; AUDA, 12-(3-adamantan-1-ylureido)dodecanoic acid; AUDA-BE, 12-(3-adamantan-1-ylureido)dodecanoic acid butyl ester; DCU, N,N′-dicyclohexylurea; ACU, N-adamantyl-N′-cyclohexylurea; AEPU, 1-adamantan-1-yl-3-{5-[2-(2-ethoxyethoxy)ethoxy]pentyl}urea; APAU, N-(1-acetylpiperidin-4-yl)-N′-(adamant-1-yl)urea; TPAU, N-(1-(2,2,2-trifluoroethanoyl)piperidin-4-yl)-N′-(adamant-1-yl)urea; AMAU, N-((1-acetylpiperidin-4-yl)methyl)-N′-(adamant-1-yl)urea; c-FCTU, 1-[4-(4-fluorophenoxy)cyclohexyl]-3-(4-trifluoromethoxyphenyl)urea; and c-FCUB, 4-{3-[4-(4-fluorophenoxy)cyclohexyl]ureido}benzoic acid.

In some embodiments, the epoxygenated fatty acid is an epoxide of linoleic acid, eicosapentaenoic acid (“EPA”) or docosahexaenoic acid (“DHA”), or a mixture thereof.

The agent can be administered orally or parenterally, as needed. For example, in some embodiments, the agent is administered systemically. In some embodiments, the agent is administered locally (e.g., topically to the site of flushing).

In some embodiments, the subject is a human.

In some embodiments, the subject has a blood concentration of niacin of at least 0.1 mM. In some embodiments, the subject is receiving a therapeutic regime of niacin.

In some embodiments, the subject is experiencing cutaneous vasodilation or flushing.

In some embodiments, the agent is co-administered with a therapeutically effective amount of niacin.

In some embodiments, the agent is co-administered with a pharmaceutical agent, other than an sEHi or an epoxygenated fatty acid, that alleviates the symptoms of niacin-induced flushing, e.g., aspirin or another non-steroidal anti-inflammatory agent, a vasoconstriction agent, laropiprant (MK-0524A), etc. The pharmaceutical agent may be co-administered in a therapeutically effective or a therapeutically ineffective (e.g., subtherapeutic or non-therapeutic) amount.

In some embodiments, the agent is co-administered with an inhibitor of transient receptor potential (TRP) channels. The TRP channel inhibitor is preferentially a selective TRP channel inhibitor. In some embodiments, the inhibitor of TRP channels is AMG9810.

In some embodiments, the increased levels of prostaglandin D₂ (“PGD2”) induced by niacin are not decreased by administration of the sEHi, the epoxygenated fatty acid or the mixture of both. In the presence of therapeutically effective levels of niacin, administration of an sEHi, an epoxygenated fatty acid or a mixture of both does not reduce or decrease PGD2 levels.

In a related aspect, the invention provides for the treatment of atherosclerosis, dyslipidemias, diabetes and related conditions by administering nicotinic acid or another nicotinic acid receptor agonist in combination with an effective amount of (i) an inhibitor of sEH, (ii) an epoxygenated fatty acid, or (iii) both an inhibitor of sEH and an epoxygenated fatty acid, such that treatment can progress without substantial flushing.

For example, in one embodiment, the invention provides methods for treating atherosclerosis in a human patient in need of such treatment comprising administering to the patient nicotinic acid or a salt or solvate thereof, or another nicotinic acid receptor agonist and (i) an inhibitor of sEH, (ii) an epoxygenated fatty acid, or (iii) both an inhibitor of sEH and an epoxygenated fatty acid, in amounts that are effective for treating atherosclerosis in the absence of substantial flushing.

In one embodiment, the invention provides methods of raising serum HDL levels in a human patient in need of such treatment, comprising administering to the patient nicotinic acid or a salt or solvate thereof, or another nicotinic acid receptor agonist and an effective amount of (i) an inhibitor of sEH, (ii) an epoxygenated fatty acid, or (iii) both an inhibitor of sEH and an epoxygenated fatty acid, said combination being effective for raising serum HDL levels in the patient in the absence of substantial flushing.

In one embodiment, the invention provides methods of treating dyslipidemia in a human patient in need of such treatment comprising administering to the patient nicotinic acid or a salt or solvate thereof, or another nicotinic acid receptor agonist and (i) an inhibitor of sEH, (ii) an epoxygenated fatty acid, or (iii) both an inhibitor of sEH and an epoxygenated fatty acid, in amounts that are effective for treating dyslipidemia in the absence of substantial flushing.

In one embodiment, the invention provides methods of reducing serum VLDL or LDL levels in a human patient in need of such treatment, comprising administering to the patient nicotinic acid or a salt or solvate thereof, or another nicotinic acid receptor agonist and (i) an inhibitor of sEH, (ii) an epoxygenated fatty acid, or (iii) both an inhibitor of sEH and an epoxygenated fatty acid, in amounts that are effective for reducing serum VLDL or LDL levels in the patient in the absence of substantial flushing.

In one embodiment, the invention provides methods of reducing serum triglyceride levels in a human patient in need of such treatment, comprising administering to the patient nicotinic acid or a salt or solvate thereof, or another nicotinic acid receptor agonist and (i) an inhibitor of sEH, (ii) an epoxygenated fatty acid, or (iii) both an inhibitor of sEH and an epoxygenated fatty acid, in amounts that are effective for reducing serum triglyceride levels in the patient in the absence of substantial flushing.

In one embodiment, the invention provides methods of reducing serum Lp (a) levels in a human patient in need of such treatment, comprising administering to the patient nicotinic acid or a salt or solvate thereof, or another nicotinic acid receptor agonist and (i) an inhibitor of sEH, (ii) an epoxygenated fatty acid, or (iii) both an inhibitor of sEH and an epoxygenated fatty acid, in amounts that are effective for reducing serum Lp (a) levels in the patient in the absence of substantial flushing. As used herein, Lp (a) refers to lipoprotein (a).

Further embodiments of the methods are as described herein.

### DEFINITIONS

Units, prefixes, and symbols are denoted in their Système International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety. Terms not defined herein have their ordinary meaning as understood by a person of skill in the art.

“cis-Epoxyeicosatrienoic acids” (“EETs”) are biomediators synthesized by cytochrome P450 epoxygenases. As discussed further in a separate section below, while the use of unmodified EETs is the most preferred, derivatives of EETs, such as amides and esters (both natural and synthetic), EETs analogs, and EETs optical isomers can all be used in the methods of the invention, both in pure form and as mixtures of these forms. For convenience of reference, the term “EETs” as used herein refers to all of these forms unless otherwise required by context.

“Epoxide hydrolases” (“EH;” EC 3.3.2.3) are enzymes in the alpha beta hydrolase fold family that add water to 3-membered cyclic ethers termed epoxides. The addition of water to the epoxides results in the corresponding 1,2-diols (Hammock, B. D. et al., in Comprehensive Toxicology: Biotransformation (Elsevier, New York), pp. 283-305 (1997); Oesch, F. Xenobiotica 3:305-340 (1972)). Four principal EH's are known: leukotriene epoxide hydrolase, cholesterol epoxide hydrolase, microsomal EH (“mEH”), and soluble EH (“sEH,” previously called cytosolic EH). The leukotriene EH acts on leukotriene A4, whereas the cholesterol EH hydrates compounds related to the 5,6-epoxide of cholesterol. The microsomal epoxide hydrolase metabolizes monosubstituted, 1,1-disubstituted, cis-1,2-disubstituted epoxides and epoxides on cyclic systems to their corresponding diols. Because of its broad substrate specificity, this enzyme is thought to play a significant role in ameliorating epoxide toxicity. Reactions of detoxification typically decrease the hydrophobicity of a compound, resulting in a more polar and thereby excretable substance.

“Soluble epoxide hydrolase” (“sEH”) is an epoxide hydrolase which in many cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). NCBI Entrez Nucleotide accession number L05779 sets forth the nucleic acid sequence encoding the protein, as well as the 5′ untranslated region and the 3′ untranslated region. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)). Soluble EH is only very distantly related to mEH and hydrates a wide range of epoxides not on cyclic systems. In contrast to the role played in the degradation of potential toxic epoxides by mEH, sEH is believed to play a role in the formation or degradation of endogenous chemical mediators. Unless otherwise specified, as used herein, the terms “soluble epoxide hydrolase” and “sEH” refer to human sEH.

Unless otherwise specified, as used herein, the terms “sEH inhibitor” (also abbreviated as “sEHI”) or “inhibitor of sEH” refer to an inhibitor of human sEH. Preferably, the inhibitor does not also inhibit the activity of microsomal epoxide hydrolase by more than 25% at concentrations at which the inhibitor inhibits sEH by at least 50%, and more preferably does not inhibit mEH by more than 10% at that concentration. For convenience of reference, unless otherwise required by context, the term “sEH inhibitor” as used herein encompasses prodrugs which are metabolized to active inhibitors of sEH. Further for convenience of reference, and except as otherwise required by context, reference herein to a compound as an inhibitor of sEH includes reference to derivatives of that compound (such as an ester of that compound) that retain activity as an sEH inhibitor.

By “physiological conditions” is meant an extracellular milieu having conditions (e.g., temperature, pH, and osmolarity) which allows for the sustenance or growth of a cell of interest.

“Micro-RNA” (“miRNA”) refers to small, noncoding RNAs of 18-25 nt in length that negatively regulate their complementary mRNAs at the posttranscriptional level in many eukaryotic organisms. See, e.g., Kurihara and Watanabe, Proc Natl Acad Sci USA 101(34):12753-12758 (2004). Micro-RNA's were first discovered in the roundworm C. elegans in the early 1990s and are now known in many species, including humans. As used herein, it refers to exogenously administered miRNA unless specifically noted or otherwise required by context.

The term “therapeutically effective amount” refers to that amount of the compound being administered sufficient to prevent or decrease the development of one or more of the symptoms of the disease, condition or disorder being treated.

The terms “prophylactically effective amount” and “amount that is effective to prevent” refer to that amount of drug that will prevent or reduce the risk of occurrence of the biological or medical event that is sought to be prevented. In many instances, the prophylactically effective amount is the same as the therapeutically effective amount.

“Subtherapeutic dose” refers to a dose of a pharmacologically active agent(s), either as an administered dose of pharmacologically active agent, or actual level of pharmacologically active agent in a subject that functionally is insufficient to elicit the intended pharmacological effect in itself (e.g., reducing or inhibiting flushing, pain or inflammation), or that quantitatively is less than the established therapeutic dose for that particular pharmacological agent (e.g., as published in a reference consulted by a person of skill, for example, doses for a pharmacological agent published in the Physicians' Desk Reference, 62nd Ed., 2008, Thomson Healthcare or Brunton, et al., Goodman & Gilman's The Pharmacological Basis of Therapeutics, 11th edition, 2006, McGraw-Hill Professional). A “subtherapeutic dose” can be defined in relative terms (i.e., as a percentage amount (less than 100%) of the amount of pharmacologically active agent conventionally administered). For example, a subtherapeutic dose amount can be about 1% to about 75% of the amount of pharmacologically active agent conventionally administered. In some embodiments, a subtherapeutic dose can be about 75%, 50%, 30%, 25%, 20%, 10% or less, than the amount of pharmacologically active agent conventionally administered.

The terms “patient,” “subject” or “individual” interchangeably refers to a mammal, for example, a human or a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster).

The terms “reduce,” “inhibit,” “relieve,” “alleviate” refer to the detectable decrease in symptoms of cutaneous vasodilation or flushing, as determined by a trained clinical observer. A reduction in cutaneous vasodilation or flushing also can be measured by self-assessment (e.g., by reporting of the patient) and by applying assays well known in the art (e.g., tests for cutaneous vasodilation). For example, assessment of cutaneous flushing can be achieved by patient reporting using a flushing symptom score as well as quantitative measurement of molar skin flow by laser Doppler perfusion imaging. In some cases, a determination of the reduction, blocking or prevention of flushing is made by visual inspection. Determination of a reduction of cutaneous vasodilation can be made by comparing patient status before and after treatment, or in comparison to an untreated control. In some embodiments, the administration of an EET or an sEHi will completely inhibit or block any detectable cutaneous vasodilation or flushing induced by niacin.

The phrase “in the absence of substantial flushing” refers to the side effect that is often seen when nicotinic acid is administered in therapeutic amounts. Flushing typically entails a reddening of the skin, accompanied by warmth, itchiness or irritation. The flushing effect of nicotinic acid usually becomes less frequent and less severe as the patient develops tolerance to the drug at therapeutic doses, but the flushing effect still occurs to some extent. Thus, “in the absence of substantial flushing” refers to the reduced severity of flushing when it occurs, or fewer flushing events than would otherwise occur. Preferably, the incidence of flushing is reduced by at least about a third, more preferably the incidence is reduced by half, and most preferably, the flushing incidence is reduced by about two thirds or more. Likewise, the severity is preferably reduced by at least about a third, more preferably by at least half, and most preferably by at least about two thirds. Blocking or achieving a one hundred percent reduction in flushing incidence and severity is most preferable, but is not required.

“Atherosclerosis” as used herein refers to a form of vascular disease characterized by the deposition of atheromatous plaques containing cholesterol and lipids on the innermost layer of the walls of large and medium-sized arteries. Atherosclerosis encompasses vascular diseases and conditions that are recognized and understood by physicians practicing in the relevant fields of medicine. Atherosclerotic cardiovascular disease, including restenosis following revascularization procedures, coronary heart disease (also known as coronary artery disease or ischemic heart disease), cerebrovascular disease including multi-infarct dementia, and peripheral vessel disease including erectile dysfunction, are all clinical manifestations of atherosclerosis and are therefore encompassed by the terms “atherosclerosis” and “atherosclerotic disease.”

“Dyslipidemia” is used in the conventional sense to refer to abnormal levels of plasma lipids, such as HDL (low), LDL (high), VLDL (high), triglycerides (high), lipoprotein (a) (high), FFA (high) and other serum lipids, or combinations thereof. It may be an uncomplicated condition or part of a particular related disease or condition such as diabetes (diabetic dyslipidemia), metabolic syndrome and the like. Thus, uncomplicated dyslipidemias as well as those that are associated with underlying conditions are treatable by the present invention.

The term “co-administered” refers to two active pharmacological agents in the blood or body tissues of a host at the same time. Co-administered agents can be concurrently administered (i.e., at the same time), or sequentially administered.

Compositions or methods “consisting essentially of” one or more recited elements include the elements specifically recited and may further include pharmacologically inactive components (e.g., excipients, vehicles), but do not include unrecited pharmacologically active agents.

### BRIEF DESCRIPTION OF THE DRAWINGS

FIG. 1 illustrates that administration of niacin alone to mice decreased plasma levels of epoxy-eicosatrienoic acids (“EETs”) as the ratio of EETs/DHETs. Administration of an sEH inhibitor (N-(1-(2,2,2-trifluoroethanoyl)piperidin-4-yl)-N′-(adamant-1-yl)urea (“TPAU”), 3 mg/kg) countered the reduction in EETs, returning the physiologic balance of EETs to DHETs. *p=0.044 vs. control; **p=0.036 vs. niacin.

FIG. 2 illustrates that administration of niacin alone to mice decreased plasma levels of total EETs, including 14,15-EETs, 8,9-EETs, 11,12-EETs. Administration of an sEH inhibitor (TPAU, 3 mg/kg) countered the reduction in EETs.

FIG. 3 illustrates that administration of niacin alone to mice did not decrease plasma levels of total DHETs, including 14,15-DHETs, 8,9-DHETs, 11,12-DHETs. These results indicate that the ratio of epoxide/diol levels decrease in niacin treated when compared to the untreated controls. This is due to a decrease in the epoxides while the diols do not change. This is then reversed following administration of the sEHI in which the EETs are stabilized. * p<0.05 vs. control; ** p<0.05 vs. niacin.

FIG. 4 illustrates that pretreatment with a concentration of TPAU that was clearly sufficient to inhibit the niacin-induced increase in tissue perfusion did not limit the increase in perfusion with administration of PGD2. Thus, inhibition of sEH, in this model, did not directly limit PGD2 induced vasodilation.

FIG. 5 illustrates that niacin administration increased PGD2 levels by approximately 100% without altering PGE2 levels. Surprisingly, administration of an sEHi (TPAU, 3 mg/kg) did not result in the expected reduction in PGD2. Rather, PGD2 levels were similar to those seen with administration of niacin alone.

FIG. 6 illustrates that niacin-induced flushing is limited in sEH knockout mice. Niacin caused an approximate 400% increase in ear perfusion that peaked at approximately 4 minutes after injection, and returned to near control levels by 15 min after injection. This increase in perfusion was limited to a 2xx % increase in sEHi KO mice (p<0.001).

FIG. 7 illustrates the dose response after niacin administration, without and with pre-treatment with the sEH inhibitor TPAU (0.01 to 1 mg/kg). Niacin-induced flushing was limited by sEH inhibitors.

FIG. 8 illustrates that chemical inhibition of sEH dose-dependently reverses flushing in mice. Inhibition of sEH eliminated flushing in this animal model, using either sEH knockout mice or pharmacologic inhibition of sEH with a potent and selective urea-based inhibitor (TPAU). This effect was achieved at very low concentrations of TPAU (Ki50 of approximately 0.01 mg/kg), with total inhibition of flushing at 0.3 mg/kg. There was a significant reduction in peak perfusion with TPAU 0.05 mg/kg to 1 mg/kg.

FIG. 9 illustrates the he composite results of experiments using 3 sEH inhibitors (TPAU, t-AUCB, and sorafenib), and well as aspirin and celecoxib. Each intervention significantly reduced peak ear tissue perfusion, without significant differences between them.

FIG. 10 illustrates that chemical inhibition of sEH reduces niacin-induced increases in products of the lipoxygenase-5 pathway.

FIG. 11 illustrates the effects of transient receptor potential (“TRP”) desensitization with repetitive capsaicin on niacin-induced flushing. Compared to the usual flushing response to niacin (open squares), desensitized animals lost the initial peak of vasodilation (closed squares). Further inhibition of sEH eliminated this second peak of vasodilation (closed dots). Data recorded manually.

FIG. 12 illustrates the effect of pharmacologic TRP blockade on PGD2-induced flushing. While PGD2 showed a rapidly rising peak (dashed line—max perfusion at 3 min), pre-treatment with the TRP channel inhibitor AMG9810 blunted the intensity and rapidity of the peak (grey line). Rapid upstroke at first acquisition time point is time of niacin injection. The data of these experiments support the hypothesis that PGD2 and TRP channel opening are involved in niacin-induced flushing. Additional experiments will define whether this is a reproducible effect. Data recorded at high temporal resolution using computerized software. Scale is 5 points/second, total duration is 400 seconds).

### DETAILED DESCRIPTION

### 1. Introduction

The enzyme “soluble epoxide hydrolase” (“sEH”) acts on an important branch of the arachidonic acid pathway degrading anti-inflammatory and analgesic metabolites. Cis-Epoxyeicosatrienoic acids” (“EETs”) are biomediators synthesized by cytochrome P450 epoxygenases, and are hydrolyzed by sEH into the corresponding diols, which are pro-inflammatory. EETs and inhibitors of sEH (“sEHI”) have been previously found to be useful as anti-inflammatories.

The present invention is based, in part, on the surprising discovery that sEHi and epoxygenated fatty acids are also useful to relieve niacin-induced cutaneous vasodilation (“flushing”). Our discovery is the first example of a novel mechanism for reducing or preventing the undesirable side effect of niacin-induced cutaneous vasodilation or flushing. Based on present understanding in the art, niacin-induced flushing is mediated by increased levels of PGD2. Surprisingly, administration of an epoxygenated fatty acid or an inhibitor of sEH prevents, reduces or blocks niacin-induced flushing without decreasing the increased levels of PGD2 elicited by therapeutic niacin administration. That is, administration of an epoxygenated fatty acid or an inhibitor of sEH in the presence of niacin does not decrease the blood, plasma or serum concentrations of PGD2. The therapeutic usefulness of an epoxygenated fatty acid or an inhibitor of sEH to reduce or prevent niacin-induced flushing is further unexpected in view of the vasodilatory properties of epoxygenated fatty acids, including EETs. For example, it is known that EETs increase endothelial derived hyperpolarizing factor (EDHF). See, Xu, et al., Proc Natl Acad Sci USA. (2006) 103(49):18733-8 and Medhora, et al., Jpn J Pharmacol. (2001) 86(4):369-75. Inhibiting and/or preventing niacin-induced flushing by administration of an epoxygenated fatty acid or an inhibitor of sEH has advantages over presently available treatments because it is efficacious in counteracting the undesirable side effect of flushing without itself introducing undesirable side effects. Also, increasing blood or serum levels of epoxygenated fatty acids, either by their direct administration or indirectly by inhibiting sEH, does not block PGD2 in the presence of therapeutic levels of niacin. The data presented herein show that pharmacologic inhibition of sEH almost completely eliminated or blocked niacin-induced flushing.

2. Patients Who can Benefit from use of Epoxygenated Fatty Acids, sEHI or Both to Reduce, Inhibit or Prevent Flushing

The present methods find use in treating, i.e., reducing, relieving, alleviating, ameliorating, inhibiting, blocking or preventing nicotinic acid-induced flushing in a subject or patient in need thereof, e.g., patients receiving nicotinic acid as part of a therapeutic regimen, e.g., to treat atherosclerosis, high cholesterol levels, raise serum HDL levels, treat dyslipidemia, reduce serum VLDL and/or LDL levels, reduce serum triglyceride levels, reduce serum Lp(a) levels, and/or manage diabetes.

Niacin or nicotinic acid (pyridine-3-carboxylic acid) is a drug commonly known for its effect in the elevation of high density lipoproteins (HDL) levels, as well as other beneficial alterations of the lipid profile (lowering very low density (VLDL), low density lipoprotein (LDL), triglycerides, free fatty acids (FFA) and lipoprotein (a) [Lp(a)]). Nicotinic acid raises HDL levels when administered to humans in therapeutically effective doses, e.g., about 50 mg to as high as about 8 grams per day. However, nicotinic acid is frequently associated with cutaneous vasodilation, also called flushing. Flushing typically entails a reddening of the skin, accompanied by warmth, itchiness or irritation. It can be extremely unpleasant, and can be so severe that many patients discontinue nicotinic acid treatment.

For methods of treating, the subject or patient will have levels of niacin in the blood, plasma or serum sufficient to induce cutaneous vasodilation or flushing. For example, the patient or subject can have concentrations of niacin in the blood, serum or plasma at or above a threshold concentration of niacin considered to be therapeutically effective, for example, above a concentration of about 0.1 mM or higher. For methods of therapeutic treatment, the patient is generally actively exhibiting symptoms of flushing.

For methods of prevention, the subject or patient will have levels of niacin in the blood, plasma or serum that are insufficient to induce cutaneous vasodilation or flushing (i.e., below about 0.1 mM). However, the patient is receiving therapeutically effective amounts of niacin as part of a treatment regimen. In cases of prevention, the patient may not be actively exhibiting flushing symptoms.

For methods of treatment or prevention, the epoxygenated fatty acid, sEHi, or both can be co-administered with niacin. For example, the epoxygenated fatty acid, sEHi, or both can be concurrently administered with niacin. Alternatively, the epoxygenated fatty acid, sEHi, or both and the niacin are administered at different times (i.e., sequentially) such that therapeutically effective concentrations of the EET, sEHi, or both and the niacin are in the blood at the same time. In some embodiments, a therapeutically effective amount of niacin is first administered and then a therapeutically effective amount of the epoxygenated fatty acid, sEHi, or mixtures thereof, is administered. In some embodiments, a therapeutically effective amount of the epoxygenated fatty acid, sEHi, or both is first administered and then a therapeutically effective amount of niacin is administered.

In some embodiments of the invention, the person being treated with epoxygenated fatty acids, sEHI, or both, does not have hypertension or is not currently being treated with an anti-hypertension agent that is an inhibitor of sEH. In some embodiments, the person being treated does not have inflammation or, if he or she has inflammation, has not been treated with an sEH inhibitor as an anti-inflammatory agent. In some preferred embodiments, the person is being treated for inflammation but by an anti-inflammatory agent, such as a steroid, that is not an inhibitor of sEH. Whether or not any particular anti-inflammatory or anti-hypertensive agent is also a sEH inhibitor can be readily determined by standard assays, such as those taught in U.S. Pat. No. 5,955,496.

In some embodiments, the patient's disease or condition is not caused by an autoimmune disease or a disorder associated with a T-lymphocyte mediated immune function autoimmune response. In some embodiments, the patient does not have a pathological condition selected from type 1 or type 2 diabetes, insulin resistance syndrome, atherosclerosis, coronary artery disease, angina, ischemia, ischemic stroke, Raynaud's disease, or renal disease.

In some embodiments, the patient is not a person with diabetes mellitus. In some embodiments, the patient is not a person whose blood pressure is 130/80 or less, a person with metabolic syndrome whose blood pressure is less than 130/85, a person with a triglyceride level over 215 mg/dL, or a person with a cholesterol level over 200 mg/dL, or is a person with one or more of these conditions who is not taking an inhibitor of sEH. In some embodiments, the patient does not have an obstructive pulmonary disease, an interstitial lung disease, or asthma.

In some embodiments, the patient is not also currently being treated with an inhibitor of one or more enzymes selected from the group consisting of cyclo-oxygenase (“COX”)-1, COX-2, and 5-lipoxygenase (“5-LOX”), or 5-lipoxygenase activating protein (“FLAP”). It is noted that many people take a daily low dose of aspirin (e.g., 81 mg) to reduce their chance of heart attack, or take an occasional aspirin to relieve a headache. Persons taking low dose aspirin to reduce the risk of heart attack are not currently known to take that aspirin in combination with an epoxygenated fatty acid or sEHI to potentiate that effect. It is also contemplated that persons taking an occasional aspirin or ibuprofen tablet to relieve a headache or other episodic minor aches or pain would not ordinarily take that tablet in combination with an epoxygenated fatty acid or sEHI to potentiate that pain relief. In some embodiments, therefore, the patient being treated by the methods of the invention may have taken an inhibitor of COX-1, COX-2, or 5-LOX in low doses, or taken such an inhibitor on an occasional basis to relieve an occasional minor ache or pain.

In some embodiments, the patient does not have dilated cardiomyopathy or arrhythmia.

In some embodiments, the patient is not applying epoxygenated fatty acids or sEHI topically for pain relief. In some embodiments, the patient is not administering epoxygenated fatty acids or sEHI topically to the eye to relieve, for example, dry eye syndrome or intraocular pressure. In some embodiments, the patient does not have glaucoma or is being treated for glaucoma with agents that do not also inhibit sEH.

In some embodiments, the patient does not suffer from anxiety, panic attacks, agitation, status epilepticus, other forms of epilepsy, symptoms of alcohol or opiate withdrawal, insomnia, or mania. In some embodiments, the patient has one of the conditions listed in the last sentence, but is not being treated for the condition with an epoxygenated fatty acid, an sEHI, or with both.

In some embodiments, the patient is not being treated for cancer of cells expressing peripheral benzodiazepine receptors (PBR) or CB₂ receptors. In some embodiments, a patient being treated for a cancer expressing such receptors is not being treated with an epoxygenated fatty acid, an sEHI, or with both.

In some embodiments, the patient is not being treated to reduce oxygen radical damage. In some embodiments, a patient being treated to reduce oxygen radical damage is not being treated with an epoxygenated fatty acid, an sEHI, or with both.

In some embodiments, the patient is not being treated for irritable bowel syndrome. In some embodiments, a patient being treated for irritable bowel syndrome is not being treated with an epoxygenated fatty acid, an sEHI, or with both.

### 3. Inhibitors of Soluble Epoxide Hydrolase

Scores of sEH inhibitors are known, of a variety of chemical structures. Derivatives in which the urea, carbamate or amide pharmacophore (as used herein, “pharmacophore” refers to the section of the structure of a ligand that binds to the sEH) is covalently bound to both an adamantane and to a 12 carbon chain dodecane are particularly useful as sEH inhibitors. Derivatives that are metabolically stable are preferred, as they are expected to have greater activity in vivo. Selective and competitive inhibition of sEH in vitro by a variety of urea, carbamate, and amide derivatives is taught, for example, by Morisseau et al., Proc. Natl. Acad. Sci. U.S. A, 96:8849-8854 (1999), which provides substantial guidance on designing urea derivatives that inhibit the enzyme.

Derivatives of urea are transition state mimetics that form a preferred group of sEH inhibitors. Within this group, N,N′-dodecyl-cyclohexyl urea (DCU), is preferred as an inhibitor, while N-cyclohexyl-N′-dodecylurea (CDU) is particularly preferred. Some compounds, such as dicyclohexylcarbodiimide (a lipophilic diimide), can decompose to an active urea inhibitor such as DCU. Any particular urea derivative or other compound can be easily tested for its ability to inhibit sEH by standard assays, such as those discussed herein. The production and testing of urea and carbamate derivatives as sEH inhibitors is set forth in detail in, for example, Morisseau et al., Proc Natl Acad Sci (USA) 96:8849-8854 (1999).

N-Adamantyl-N′-dodecyl urea (“ADU”) is both metabolically stable and has particularly high activity on sEH. (Both the 1- and the 2-adamantyl ureas have been tested and have about the same high activity as an inhibitor of sEH.) Thus, isomers of adamantyl dodecyl urea are preferred inhibitors. It is further expected that N,N′-dodecyl-cyclohexyl urea (DCU), and other inhibitors of sEH, and particularly dodecanoic acid ester derivatives of urea, are suitable for use in the methods of the invention. Preferred inhibitors include:

12-(3-Adamantan-1-yl-ureido)dodecanoic acid (AUDA),

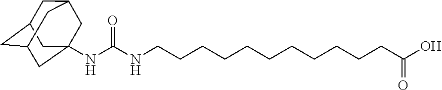

12-(3-Adamantan-1-yl-ureido)dodecanoic acid butyl ester (AUDA-BE),

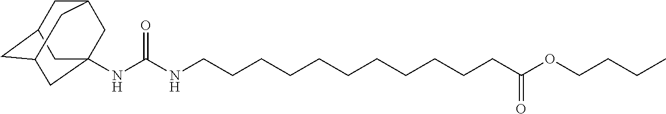

Adamantan-1-yl-3-{5-[2-(2-ethoxyethoxy)ethoxy]pentyl}urea (compound 950, also referred to herein as “AEPU”), and

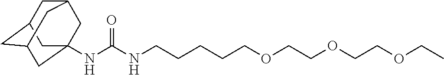

Another preferred group of inhibitors are piperidines. The following Table sets forth some exemplar piperidines and their ability to inhibit sEH activity, expressed as the amount needed to reduce the activity of the enzyme by 50% (expressed as “IC₅₀”).

TABLE 1
IC₅₀ values for selected alkylpiperidine-based sEH inhibitors

| | | 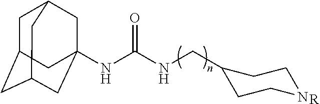 | | | |
|---|---|---|---|---|---|
| | | n = 0 | | n = 1 | |
| | | Compound | IC₅₀ (μM)^{a} | Compound | IC₅₀ (μM)^{a} |
| R: | H | I | 0.30 | II | 4.2 |
| | 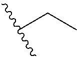 | 3a | 3.8 | 4.a | 3.9 |
| | 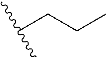 | 3b | 0.81 | 4b | 2.6 |
| | 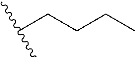 | 3c | 1.2 | 4c | 0.61 |
| | 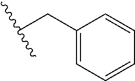 | 3d | 0.01 | 4d | 0.11 |

^{a}As determined via a kinetic fluorescent assay.

A number of other sEH inhibitors which can be used in the methods and compositions of the invention are set forth in co-owned applications PCT/US2008/072199, PCT/US2007/006412, PCT/US2005/038282, PCT/US2005/08765, PCT/US2004/010298 and U.S. Published Patent Application Publication 2005/0026844, each of which is hereby incorporated herein by reference in its entirety for all purposes.

U.S. Pat. No. 5,955,496 (the '496 patent) also sets forth a number of sEH inhibitors which can be used in the methods of the invention. One category of these inhibitors comprises inhibitors that mimic the substrate for the enzyme. The lipid alkoxides (e.g., the 9-methoxide of stearic acid) are an exemplar of this group of inhibitors. In addition to the inhibitors discussed in the '496 patent, a dozen or more lipid alkoxides have been tested as sEH inhibitors, including the methyl, ethyl, and propyl alkoxides of oleic acid (also known as stearic acid alkoxides), linoleic acid, and arachidonic acid, and all have been found to act as inhibitors of sEH.

In another group of embodiments, the '496 patent sets forth sEH inhibitors that provide alternate substrates for the enzyme that are turned over slowly. Exemplars of this category of inhibitors are phenyl glycidols (e.g., S,S-4-nitrophenylglycidol), and chalcone oxides. The '496 patent notes that suitable chalcone oxides include 4-phenylchalcone oxide and 4-fluorochalcone oxide. The phenyl glycidols and chalcone oxides are believed to form stable acyl enzymes.

Additional inhibitors of sEH suitable for use in the methods of the invention are set forth in U.S. Pat. Nos. 6,150,415 (the '415 patent) and 6,531,506 (the '506 patent). Two preferred classes of sEH inhibitors of the invention are compounds of Formulas 1 and 2, as described in the '415 and '506 patents. Means for preparing such compounds and assaying desired compounds for the ability to inhibit epoxide hydrolases are also described. The '506 patent, in particular, teaches scores of inhibitors of Formula 1 and some twenty sEH inhibitors of Formula 2, which were shown to inhibit human sEH at concentrations as low as 0.1 μM. Any particular sEH inhibitor can readily be tested to determine whether it will work in the methods of the invention by standard assays. Esters and salts of the various compounds discussed above or in the cited patents, for example, can be readily tested by these assays for their use in the methods of the invention.

As noted above, chalcone oxides can serve as an alternate substrate for the enzyme. While chalcone oxides have half lives which depend in part on the particular structure, as a group the chalcone oxides tend to have relatively short half lives (a drug's half life is usually defined as the time for the concentration of the drug to drop to half its original value. See, e.g., Thomas, G., Medicinal Chemistry: an introduction, John Wiley & Sons Ltd. (West Sussex, England, 2000)). Since the various uses of the invention contemplate inhibition of sEH over differing periods of time which can be measured in days, weeks, or months, chalcone oxides, and other inhibitors which have a half life whose duration is shorter than the practitioner deems desirable, are preferably administered in a manner which provides the agent over a period of time. For example, the inhibitor can be provided in materials that release the inhibitor slowly. Methods of administration that permit high local concentrations of an inhibitor over a period of time are known, and are not limited to use with inhibitors which have short half lives although, for inhibitors with a relatively short half life, they are a preferred method of administration.

In addition to the compounds in Formula 1 of the '506 patent, which interact with the enzyme in a reversible fashion based on the inhibitor mimicking an enzyme-substrate transition state or reaction intermediate, one can have compounds that are irreversible inhibitors of the enzyme. The active structures such as those in the Tables or Formula 1 of the '506 patent can direct the inhibitor to the enzyme where a reactive functionality in the enzyme catalytic site can form a covalent bond with the inhibitor. One group of molecules which could interact like this would have a leaving group such as a halogen or tosylate which could be attacked in an SN2 manner with a lysine or histidine. Alternatively, the reactive functionality could be an epoxide or Michael acceptor such as an α/β-unsaturated ester, aldehyde, ketone, ester, or nitrile.

Further, in addition to the Formula 1 compounds, active derivatives can be designed for practicing the invention. For example, dicyclohexyl thio urea can be oxidized to dicyclohexylcarbodiimide which, with enzyme or aqueous acid (physiological saline), will form an active dicyclohexylurea. Alternatively, the acidic protons on carbamates or ureas can be replaced with a variety of substituents which, upon oxidation, hydrolysis or attack by a nucleophile such as glutathione, will yield the corresponding parent structure. These materials are known as prodrugs or protoxins (Gilman et al., The Pharmacological Basis of Therapeutics, 7th Edition, MacMillan Publishing Company, New York, p. 16 (1985)) Esters, for example, are common prodrugs which are released to give the corresponding alcohols and acids enzymatically (Yoshigae et al., Chirality, 9:661-666 (1997)). The drugs and prodrugs can be chiral for greater specificity. These derivatives have been extensively used in medicinal and agricultural chemistry to alter the pharmacological properties of the compounds such as enhancing water solubility, improving formulation chemistry, altering tissue targeting, altering volume of distribution, and altering penetration. They also have been used to alter toxicology profiles.

There are many prodrugs possible, but replacement of one or both of the two active hydrogens in the ureas described here or the single active hydrogen present in carbamates is particularly attractive. Such derivatives have been extensively described by Fukuto and associates. These derivatives have been extensively described and are commonly used in agricultural and medicinal chemistry to alter the pharmacological properties of the compounds. (Black et al., Journal of Agricultural and Food Chemistry, 21(5):747-751 (1973); Fahmy et al, Journal of Agricultural and Food Chemistry, 26(3):550-556 (1978); Jojima et al., Journal of Agricultural and Food Chemistry, 31(3):613-620 (1983); and Fahmy et al., Journal of Agricultural and Food Chemistry, 29(3):567-572 (1981).)

Such active proinhibitor derivatives are within the scope of the present invention, and the just-cited references are incorporated herein by reference. Without being bound by theory, it is believed that suitable inhibitors of the invention mimic the enzyme transition state so that there is a stable interaction with the enzyme catalytic site. The inhibitors appear to form hydrogen bonds with the nucleophilic carboxylic acid and a polarizing tyrosine of the catalytic site.

In some embodiments, the sEH inhibitor used in the methods taught herein is a “soft drug.” Soft drugs are compounds of biological activity that are rapidly inactivated by enzymes as they move from a chosen target site. EETs and simple biodegradable derivatives administered to an area of interest may be considered to be soft drugs in that they are likely to be enzymatically degraded by sEH as they diffuse away from the site of interest following administration. Some sEHI, however, may diffuse or be transported following administration to regions where their activity in inhibiting sEH may not be desired. Thus, multiple soft drugs for treatment have been prepared. These include but are not limited to carbamates, esters, carbonates and amides placed in the sEHI, approximately 7.5 angstroms from the carbonyl of the central pharmacophore. These are highly active sEHI that yield biologically inactive metabolites by the action of esterase and/or amidase. Groups such as amides and carbamates on the central pharmacophores can also be used to increase solubility for applications in which that is desirable in forming a soft drug. Similarly, easily metabolized ethers may contribute soft drug properties and also increase the solubility.

In some embodiments, sEH inhibition can include the reduction of the amount of sEH. As used herein, therefore, sEH inhibitors can therefore encompass nucleic acids that inhibit expression of a gene encoding sEH. Many methods of reducing the expression of genes, such as reduction of transcription and siRNA, are known, and are discussed in more detail below.

Preferably, the inhibitor inhibits sEH without also significantly inhibiting microsomal epoxide hydrolase (“mEH”). Preferably, at concentrations of 500 μM, the inhibitor inhibits sEH activity by at least 50% while not inhibiting mEH activity by more than 10%. Preferred compounds have an IC₅₀ (inhibition potency or, by definition, the concentration of inhibitor which reduces enzyme activity by 50%) of less than about 500 μM. Inhibitors with IC₅₀s of less than 500 μM are preferred, with IC₅₀s of less than 100 μM being more preferred and, in order of increasing preference, an IC₅₀ of 50 μM, 40 μM, 30 μM, 25 μM, 20 μM, 15 μM, 10 μM, 5 μM, 3 μM, 2 μM, 1 μM or even less being still more preferred. Assays for determining sEH activity are known in the art and described elsewhere herein.

### 4. Epoxygenated Fatty Acids

In some embodiments, an epoxygenated fatty acid is administered. The epoxygenated fatty acid can be co-administered with niacin. The epoxygenated fatty acid can be co-administered with an sEH inhibitor. Exemplary epoxygenated fatty acids include epoxides of linoleic acid, eicosapentaenoic acid (“EPA”) and docosahexaenoic acid (“DHA”).

The fatty acids eicosapentaenoic acid (“EPA”) and docosahexaenoic acid (“DHA”) have recently become recognized as having beneficial effects, and fish oil tablets, which are a good source of these fatty acids, are widely sold as supplements. In 2003, it was reported that these fatty acids reduced pain and inflammation. Sethi, S. et al., Blood 100: 1340-1346 (2002). The paper did not identify the mechanism of action, nor the agents responsible for this relief.

Cytochrome P450 (“CYP450”) metabolism produces cis-epoxydocosapentanoic acids (“EpDPEs”) and cis-epoxyeicosatetraenoic acids (“EpETEs”) from docosahexaenoic acid (“DHA”) and eicosapentaenoic acid (“EPA”), respectively. These epoxides are known endothelium-derived hyperpolarizing factors (“EDHFs”). These EDHFs, and others yet unidentified, are mediators released from vascular endothelial cells in response to acetylcholine and bradykinin, and are distinct from the NOS- (nitric oxide) and COX-derived (prostacyclin) vasodilators. Overall cytochrome P450 (CYP450) metabolism of polyunsaturated fatty acids produces epoxides, such as EETs, which are prime candidates for the active mediator(s). 14(15)-EpETE, for example, is derived via epoxidation of the 14,15-double bond of EPA and is the ω-3 homolog of 14(15)-EpETrE (“14(15)EET”) derived via epoxidation of the 14,15-double bond of arachidonic acid.

As mentioned, we have found that it is beneficial to elevate the levels of EETs, which are epoxides of the fatty acid arachidonic acid. Our studies of the effects of EETs has led us to realization that the anti-flushing effect of EPA and DHA are likely due to increasing the levels of the epoxides of these two fatty acids. Thus, we expect that increasing the levels of epoxides of EPA, of DHA, or of both, will act to reduce flushing in mammals in need thereof. This beneficial effect of the epoxides of these fatty acids has not been previously recognized. Moreover, these epoxides have not previously been administered as agents, in part because, as noted above, epoxides have generally been considered too labile to be administered.

Like EETs, the epoxides of EPA and DHA are substrates for sEH. The epoxides of EPA and DHA are produced in the body at low levels by the action of cytochrome P450s. Endogenous levels of these epoxides can be maintained or increased by the administration of sEHI. However, the endogenous production of these epoxides is low and usually occurs in relatively special circumstances, such as the resolution of inflammation. Our expectation is that administering these epoxides from exogenous sources will aid in the prevention and reduction or inhibition of flushing. We further expect that it will be beneficial to counteract flushing to inhibit sEH with sEHI to reduce hydrolysis of these epoxides, thereby maintaining them at relatively high levels.

EPA has five unsaturated bonds, and thus five positions at which epoxides can be formed, while DHA has six. The epoxides of EPA are typically abbreviated and referred to generically as “EpETEs”, while the epoxides of DHA are typically abbreviated and referred to generically as “EpDPEs”. The specific regioisomers of the epoxides of each fatty acid are set forth in the following Table:

TABLE A
Regioisomers of Eicosapentaenoic acid (“EPA”) epoxides:

```
1. Formal name: (±)5(6)-epoxy-8Z,11Z,14Z,17Z-eicosatetraenoic acid,
    Synonym 5(6)-epoxy Eicosatetraenoic acid
    Abbreviation 5(6)-EpETE
2. Formal name: (±)8(9)-epoxy-5Z,11Z,14Z,17Z-eicosatetraenoic acid,
    Synonym 8(9)-epoxy Eicosatetraenoic acid
    Abbreviation 8(9)-EpETE
3. Formal name: (±)11(12)-epoxy-5Z,8Z,14Z,17Z-eicosatetraenoic acid,
    Synonym 11(12)-epoxy Eicosatetraenoic acid
    Abbreviation 11(12)-EpETE
4. Formal name: (±)14(15)-epoxy-5Z,8Z,11Z,17Z-eicosatetraenoic acid,
    Synonym 14(15)-epoxy Eicosatetraenoic acid
    Abbreviation 14(15)-EpETE
5. Formal name: (±)17(18)-epoxy-5Z,8Z,11Z,14Z-eicosatetraenoic acid,
    Synonym 17(18)-epoxy Eicosatetraenoic acid
    Abbreviation 17(18)-EpETE
```

Regioisomers of Docosahexaenoic acid (“DHA”) epoxides:

```
1. Formal name: (±) 4(5)-epoxy-7Z,10Z,13Z,16Z,19Z-docosapentaenoic
  acid,
    Synonym 4(5)-epoxy Docosapentaenoic acid
    Abbreviation 4(5)-EpDPE
2. Formal name: (±) 7(8)-epoxy-4Z,10Z,13Z,16Z,19Z-docosapentaenoic
  acid,
    Synonym 7(8)-epoxy Docosapentaenoic acid
    Abbreviation 7(8)-EpDPE
3. Formal name: (±)10(11)-epoxy-4Z,7Z,13Z,16Z,19Z-docosapentaenoic
  acid,
    Synonym 10(11)-epoxy Docosapentaenoic acid
    Abbreviation 10(11)-EpDPE
4. Formal name: (±)13(14)-epoxy-4Z,7Z,10Z,16Z,19Z-docosapentaenoic
  acid,
    Synonym 13(14)-epoxy Docosapentaenoic acid
    Abbreviation 13(14)-EpDPE
5. Formal name: (±) 16(17)-epoxy-4Z,7Z,10Z,13Z,19Z-docosapentaenoic
  acid,
    Synonym 16(17)-epoxy Docosapentaenoic acid
    Abbreviation 16(17)-EpDPE
6. Formal name: (±) 19(20)-epoxy-4Z,7Z,10Z,13Z,16Z-docosapentaenoic
  acid,
    Synonym 19(20)-epoxy Docosapentaenoic acid
    Abbreviation 19(20)-EpDPE
```

Any of these epoxides, or combinations of any of these, can be administered in the compositions and methods of the invention.

### 5. Cis-Epoxyeicosantrienoic Acids (“EETs”)

In some embodiments, the epoxygenated fatty acid is an EET. EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into dihydroxyeicosatrienoic acids (“DHETs”) is reduced. An EET can be co-administered with niacin and/or and inhibitor of sEH.

It has long been believed that EETs administered systemically would be hydrolyzed too quickly by endogenous sEH to be helpful. For example, in one prior report of EETs administration, EETs were administered by catheters inserted into mouse aortas. The EETs were infused continuously during the course of the experiment because of concerns over the short half life of the EETs. See, Liao and Zeldin, International Publication WO 01/10438 (hereafter “Liao and Zeldin”). It also was not known whether endogenous sEH could be inhibited sufficiently in body tissues to permit administration of exogenous EET to result in increased levels of EETs over those normally present. Further, it was thought that EETs, as epoxides, would be too labile to survive the storage and handling necessary for therapeutic use.

Studies from the laboratory of the present inventors, however, showed that systemic administration of EETs in conjunction with inhibitors of sEH had better results than did administration of sEH inhibitors alone. EETs were not administered by themselves in these studies since it was anticipated they would be degraded too quickly to have a useful effect. Additional studies from the laboratory of the present inventors have since shown, however, that administration of EETs by themselves has had therapeutic effect. Without wishing to be bound by theory, it is surmised that the exogenous EET overwhelms endogenous sEH, and allows EETs levels to be increased for a sufficient period of time to have therapeutic effect. Thus, EETs can be administered without also administering an sEHI to provide a therapeutic effect. Moreover, we have found that EETs, if not exposed to acidic conditions or to sEH are stable and can withstand reasonable storage, handling and administration.

In short, sEHI, EETs, or co-administration of sEHIs and of EETs, can be used in the methods of the present invention. In some embodiments, one or more EETs are administered to the patient without also administering an sEHI. In some embodiments, one or more EETs are administered shortly before or concurrently with administration of an sEH inhibitor to slow hydrolysis of the EET or EETs. In some embodiments, one or more EETs are administered after administration of an sEH inhibitor, but before the level of the sEHI has diminished below a level effective to slow the hydrolysis of the EETs.

EETs useful in the methods of the present invention include 14,15-EET, 8,9-EET and 11,12-EET, and 5,6 EETs. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

If desired, EETs, analogs, or derivatives that retain activity can be used in place of or in combination with unmodified EETs. Liao and Zeldin, supra, define EET analogs as compounds with structural substitutions or alterations in an EET, and include structural analogs in which one or more EET olefins are removed or replaced with acetylene or cyclopropane groups, analogs in which the epoxide moiety is replaced with oxitane or furan rings and heteroatom analogs. In other analogs, the epoxide moiety is replaced with ether, alkoxides, difluorocyclopropane, or carbonyl, while in others, the carboxylic acid moiety is replaced with a commonly used mimic, such as a nitrogen heterocycle, a sulfonamide, or another polar functionality. In preferred forms, the analogs or derivatives are relatively stable as compared to an unmodified EET because they are more resistant than an unmodified EET to sEH and to chemical breakdown. “Relatively stable” means the rate of hydrolysis by sEH is at least 25% less than the hydrolysis of the unmodified EET in a hydrolysis assay, and more preferably 50% or more lower than the rate of hydrolysis of an unmodified EET. Liao and Zeldin show, for example, episulfide and sulfonamide EETs derivatives. Amide and ester derivatives of EETs and that are relatively stable are preferred embodiments. In preferred forms, the analogs or derivatives have the biological activity of the unmodified EET regioisomer from which it is modified or derived in binding to the CB2 or peripheral BZD receptor. Whether or not a particular EET analog or derivative has the biological activity of the unmodified EET can be readily determined by using it in standard assays, such as radio-ligand competition assays to measure binding to the relevant receptor. As mentioned in the Definition section, above, for convenience of reference, the term “EETs” as used herein refers to unmodified EETs, and EETs analogs and derivatives unless otherwise required by context.

In some embodiments, the EET or EETs are embedded or otherwise placed in a material that releases the EET over time. Materials suitable for promoting the slow release of compositions such as EETs are known in the art. Optionally, one or more sEH inhibitors may also be placed in the slow release material.

Conveniently, the EET or EETs can be administered orally. Since EETs are subject to degradation under acidic conditions, EETs intended for oral administration can be coated with a coating resistant to dissolving under acidic conditions, but which dissolve under the mildly basic conditions present in the intestines. Suitable coatings, commonly known as “enteric coatings” are widely used for products, such as aspirin, which cause gastric distress or which would undergo degradation upon exposure to gastric acid. By using coatings with an appropriate dissolution profile, the coated substance can be released in a chosen section of the intestinal tract. For example, a substance to be released in the colon is coated with a substance that dissolves at pH 6.5-7, while substances to be released in the duodenum can be coated with a coating that dissolves at pH values over 5.5. Such coatings are commercially available from, for example, Rohm Specialty Acrylics (Rohm America LLC, Piscataway, N.J.) under the trade name “Eudragit®”. The choice of the particular enteric coating is not critical to the practice of the invention.

### 6. Assays for Epoxide Hydrolase Activity

Any of a number of standard assays for determining epoxide hydrolase activity can be used to determine inhibition of sEH. For example, suitable assays are described in Gill., et al., Anal Biochem 131:273-282 (1983); and Borhan, et al., Analytical Biochemistry 231:188-200 (1995)). Suitable in vitro assays are described in Zeldin et al., J. Biol. Chem. 268:6402-6407 (1993). Suitable in vivo assays are described in Zeldin et al., Arch Biochem Biophys 330:87-96 (1996). Assays for epoxide hydrolase using both putative natural substrates and surrogate substrates have been reviewed (see, Hammock, et al. In: Methods in Enzymology, Volume III, Steroids and Isoprenoids, Part B, (Law, J. H. and H. C. Rilling, eds. 1985), Academic Press, Orlando, Fla., pp. 303-311 and Wixtrom et al., In: Biochemical Pharmacology and Toxicology, Vol. 1: Methodological Aspects of Drug Metabolizing Enzymes, (Zakim, D. and D. A. Vessey, eds. 1985), John Wiley & Sons, Inc., New York, pp. 1-93. Several spectral based assays exist based on the reactivity or tendency of the resulting diol product to hydrogen bond (see, e.g., Wixtrom, supra, and Hammock. Anal. Biochem. 174:291-299 (1985) and Dietze, et al. Anal. Biochem. 216:176-187 (1994)).

The enzyme also can be detected based on the binding of specific ligands to the catalytic site which either immobilize the enzyme or label it with a probe such as dansyl, fluoracein, luciferase, green fluorescent protein or other reagent. The enzyme can be assayed by its hydration of EETs, its hydrolysis of an epoxide to give a colored product as described by Dietze et al., 1994, supra, or its hydrolysis of a radioactive surrogate substrate (Borhan et al., 1995, supra). The enzyme also can be detected based on the generation of fluorescent products following the hydrolysis of the epoxide. Numerous methods of epoxide hydrolase detection have been described (see, e.g., Wixtrom, supra).

The assays are normally carried out with a recombinant enzyme following affinity purification. They can be carried out in crude tissue homogenates, cell culture or even in vivo, as known in the art and described in the references cited above.

### 7. Other Means of Inhibiting sEH Activity

Other means of inhibiting sEH activity or gene expression can also be used in the methods of the invention. For example, a nucleic acid molecule complementary to at least a portion of the human sEH gene can be used to inhibit sEH gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through a mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

“RNA interference,” a form of post-transcriptional gene silencing (“PTGS”), describes effects that result from the introduction of double-stranded RNA into cells (reviewed in Fire, A. Trends Genet. 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of sEH, siRNAs to the gene encoding sEH can be specifically designed using computer programs. The cloning, sequence, and accession numbers of the human sEH sequence are set forth in Beetham et al., Arch. Biochem. Biophys. 305(1):197-201 (1993). An exemplary amino acid sequence of human sEH (GenBank Accession No. L05779; SEQ ID NO:1) and an exemplary nucleotide sequence encoding that amino acid sequence (GenBank Accession No. AAA02756; SEQ ID NO:2) are set forth in U.S. Pat. No. 5,445,956. The nucleic acid sequence of human sEH is also published as GenBank Accession No. NM_{—}001979.4; the amino acid sequence of human sEH is also published as GenBank Accession No. NP_{—}001970.2.

A program, siDESIGN from Dharmacon, Inc. (Lafayette, Colo.), permits predicting siRNAs for any nucleic acid sequence, and is available on the World Wide Web at dharmacon.com. Programs for designing siRNAs are also available from others, including Genscript (available on the Web at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

For example, using the program available from the Whitehead Institute, the following sEH target sequences and siRNA sequences can be generated:

| 1) Target: | |
|---|---|
| CAGTGTTCATTGGCCATGACTGG | (SEQ ID NO: 3) |
| Sense-siRNA: | |
| 5′-GUGUUCAUUGGCCAUGACUTT-3′ | (SEQ ID NO: 4) |
| Antisense-siRNA: | |
| 5′-AGUCAUGGCCAAUGAACACTT-3′ | (SEQ ID NO: 5) |
| 2) Target: | |
| GAAAGGCTATGGAGAGTCATCTG | (SEQ ID NO: 6) |
| Sense-siRNA: | |
| 5′-AAGGCUAUGGAGAGUCAUCTT-3′ | (SEQ ID NO: 7) |
| Antisense-siRNA: | |
| 5′-GAUGACUCUCCAUAGCCUUTT-3′ | (SEQ ID NO: 8) |
| 3) Target | |
| AAAGGCTATGGAGAGTCATCTGC | (SEQ ID NO: 9) |
| Sense-siRNA: | |
| 5′-AGGCUAUGGAGAGUCAUCUTT-3′ | (SEQ ID NO: 10) |
| Antisense-siRNA: | |
| 5′-AGAUGACUCUCCAUAGCCUTT-3′ | (SEQ ID NO: 11) |
| 4) Target: | |
| CAAGCAGTGTTCATTGGCCATGA | (SEQ ID NO: 12) |
| Sense-siRNA: | |
| 5′-AGCAGUGUUCAUUGGCCAUTT-3′ | (SEQ ID NO: 13 |
| Antisense-siRNA: | |
| 5′-AUGGCCAAUGAACACUGCUTT-3′ | (SEQ ID NO: 14 |
| 5) Target: | |
| CAGCACATGGAGGACTGGATTCC | (SEQ ID NO: 15) |
| Sense-siRNA: | |
| 5′-GCACAUGGAGGACUGGAUUTT-3′ | (SEQ ID NO: 16) |
| Antisense-siRNA: | |
| 5′-AAUCCAGUCCUCCAUGUGCTT-3′ | (SEQ ID NO: 17) |

Alternatively, siRNA can be generated using kits which generate siRNA from the gene. For example, the “Dicer siRNA Generation” kit (catalog number T510001, Gene Therapy Systems, Inc., San Diego, Calif.) uses the recombinant human enzyme “dicer” in vitro to cleave long double stranded RNA into 22 bp siRNAs. By having a mixture of siRNAs, the kit permits a high degree of success in generating siRNAs that will reduce expression of the target gene. Similarly, the Silencer™ siRNA Cocktail Kit (RNase III) (catalog no. 1625, Ambion, Inc., Austin, Tex.) generates a mixture of siRNAs from dsRNA using RNase III instead of dicer. Like dicer, RNase III cleaves dsRNA into 12-30 bp dsRNA fragments with 2 to 3 nucleotide 3′ overhangs, and 5′-phosphate and 3′-hydroxyl termini. According to the manufacturer, dsRNA is produced using T7 RNA polymerase, and reaction and purification components included in the kit. The dsRNA is then digested by RNase III to create a population of siRNAs. The kit includes reagents to synthesize long dsRNAs by in vitro transcription and to digest those dsRNAs into siRNA-like molecules using RNase III. The manufacturer indicates that the user need only supply a DNA template with opposing T7 phage polymerase promoters or two separate templates with promoters on opposite ends of the region to be transcribed.

The siRNAs can also be expressed from vectors. Typically, such vectors are administered in conjunction with a second vector encoding the corresponding complementary strand. Once expressed, the two strands anneal to each other and form the functional double stranded siRNA. One exemplar vector suitable for use in the invention is pSuper, available from OligoEngine, Inc. (Seattle, Wash.). In some embodiments, the vector contains two promoters, one positioned downstream of the first and in antiparallel orientation. The first promoter is transcribed in one direction, and the second in the direction antiparallel to the first, resulting in expression of the complementary strands. In yet another set of embodiments, the promoter is followed by a first segment encoding the first strand, and a second segment encoding the second strand. The second strand is complementary to the palindrome of the first strand. Between the first and the second strands is a section of RNA serving as a linker (sometimes called a “spacer”) to permit the second strand to bend around and anneal to the first strand, in a configuration known as a “hairpin.”

The formation of hairpin RNAs, including use of linker sections, is well known in the art. Typically, an siRNA expression cassette is employed, using a Polymerase III promoter such as human U6, mouse U6, or human H1. The coding sequence is typically a 19-nucleotide sense siRNA sequence linked to its reverse complementary antisense siRNA sequence by a short spacer. Nine-nucleotide spacers are typical, although other spacers can be designed. For example, the Ambion website indicates that its scientists have had success with the spacer TTCAAGAGA (SEQ ID NO:18). Further, 5-6 T's are often added to the 3′ end of the oligonucleotide to serve as a termination site for Polymerase III. See also, Yu et al., Mol Ther 7(2):228-36 (2003); Matsukura et al., Nucleic Acids Res 31(15):e77 (2003).

As an example, the siRNA targets identified above can be targeted by hairpin siRNA as follows. To attack the same targets by short hairpin RNAs, produced by a vector (permanent RNAi effect), sense and antisense strand can be put in a row with a loop forming sequence in between and suitable sequences for an adequate expression vector to both ends of the sequence. The following are non-limiting examples of hairpin sequences that can be cloned into the pSuper vector:

1) Target:
(SEQ ID NO: 19)
CAGTGTTCATTGGCCATGACTGG
Sense strand:
(SEQ ID NO: 20)
5′-GATCCCCGTGTTCATTGGCCATGACTTTCAAGAGAAGTCATGGCCAA
TGAACACTTTTT-3′
Antisense strand:
(SEQ ID NO: 21)
5′-AGCTAAAAAGTGTTCATTGGCCATGACTTCTCTTGAAAGTCATGGCC
AATGAACACGGG-3′
2) Target:
(SEQ ID NO: 22)
GAAAGGCTATGGAGAGTCATCTG
Sense strand:
(SEQ ID NO: 23)
5′-GATCCCCAAGGCTATGGAGAGTCATCTTCAAGAGAGATGACTCTCCA
TAGCCTTTTTTT-3′
Antisense strand:
(SEQ ID NO: 24)
5′-AGCTAAAAAAAGGCTATGGAGAGTCATCTCTCTTGAAGATGACTCTC
CATAGCCTTGGG-3′
3) Target:
(SEQ ID NO: 25)
AAAGGCTATGGAGAGTCATCTGC
Sense strand:
(SEQ ID NO: 26)
5′-GATCCCCAGGCTATGGAGAGTCATCTTTCAAGAGAAGATGACTCTCC
ATAGCCTTTTTT-3′
Antisense strand:
(SEQ ID NO: 27)
5′-AGCTAAAAAAGGCTATGGAGAGTCATCATCTCTTGAAAGATGACTCT
CCATAGCCTGGG-3′
4) Target:
(SEQ ID NO: 28)
CAAGCAGTGTTCATTGGCCATGA
Sense strand:
(SEQ ID NO: 29)
5′-GATCCCCAGCAGTGTTCATTGGCCATTTCAAGAGAATGGCCAATGAA
CACTGCTTTTTT-3′
Antisense strand:
(SEQ ID NO: 30)
5′-AGCTAAAAAAGCAGTGTTCATTGGCCATTCTCTTGAAATGGCCAATG
AACACTGCTGGG-3′
5) Target:
(SEQ ID NO: 31)
CAGCACATGGAGGACTGGATTCC
Sense strand
(SEQ ID NO: 32)
5′-GATCCCCGCACATGGAGGACTGGATTTTCAAGAGAAATCCAGTCCTC
CATGTGCTTTTT-3′
Antisense strand:
(SEQ ID NO: 33)
5′-AGCTAAAAAGCACATGGAGGACTGGATTTCTCTTGAAAATCCAGTCC
TCCATGTGCGGG-3′

In addition to siRNAs, other means are known in the art for inhibiting the expression of antisense molecules, ribozymes, and the like are well known to those of skill in the art. The nucleic acid molecule can be a DNA probe, a riboprobe, a peptide nucleic acid probe, a phosphorothioate probe, or a 2′-O methyl probe.

Generally, to assure specific hybridization, the antisense sequence is substantially complementary to the target sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to the sEH gene is retained as a functional property of the polynucleotide. In one embodiment, the antisense molecules form a triple helix-containing, or “triplex” nucleic acid. Triple helix formation results in inhibition of gene expression by, for example, preventing transcription of the target gene (see, e.g., Cheng et al., 1988, J. Biol. Chem. 263:15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83:9591)

Antisense molecules can be designed by methods known in the art. For example, Integrated DNA Technologies (Coralville, Iowa) makes available a program found on the worldwide web “biotools.idtdna.com/antisense/AntiSense.aspx”, which will provide appropriate antisense sequences for nucleic acid sequences up to 10,000 nucleotides in length. Using this program with the sEH gene provides the following exemplar sequences:

| | |
|---|---|
| 1) UGUCCAGUGCCCACAGUCCU | (SEQ ID NO: 34) |
| 2) UUCCCACCUGACACGACUCU | (SEQ ID NO: 35) |
| 3) GUUCAGCCUCAGCCACUCCU | (SEQ ID NO: 36) |
| 4) AGUCCUCCCGCUUCACAGA | (SEQ ID NO: 37) |
| 5) GCCCACUUCCAGUUCCUUUCC | (SEQ ID NO: 38) |

In another embodiment, ribozymes can be designed to cleave the mRNA at a desired position. (See, e.g., Cech, 1995, Biotechnology 13:323; and Edgington, 1992, Biotechnology 10:256 and Hu et al., PCT Publication WO 94/03596).

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules of the invention may be prepared by de novo chemical synthesis or by cloning. For example, an antisense RNA can be made by inserting (ligating) a sEH gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

It will be appreciated that the oligonucleotides can be made using nonstandard bases (e.g., other than adenine, cytidine, guanine, thymine, and uridine) or nonstandard backbone structures to provides desirable properties (e.g., increased nuclease-resistance, tighter-binding, stability or a desired Tm). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, phosphoramidates.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996). Another subsequence, the h (hydrophobic) domain of signal peptides, was found to have similar cell membrane translocation characteristics (see, e.g., Lin et al., J. Biol. Chem. 270:14255-14258 (1995)). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

More recently, it has been discovered that siRNAs can be introduced into mammals without eliciting an immune response by encapsulating them in nanoparticles of cyclodextrin. Information on this method can be found on the worldwide web at “nature.com/news/2005/050418/full/050418-6.html.”

In another method, the nucleic acid is introduced directly into superficial layers of the skin or into muscle cells by a jet of compressed gas or the like. Methods for administering naked polynucleotides are well known and are taught, for example, in U.S. Pat. No. 5,830,877 and International Publication Nos. WO 99/52483 and 94/21797. Devices for accelerating particles into body tissues using compressed gases are described in, for example, U.S. Pat. Nos. 6,592,545, 6,475,181, and 6,328,714. The nucleic acid may be lyophilized and may be complexed, for example, with polysaccharides to form a particle of appropriate size and mass for acceleration into tissue. Conveniently, the nucleic acid can be placed on a gold bead or other particle which provides suitable mass or other characteristics. Use of gold beads to carry nucleic acids into body tissues is taught in, for example, U.S. Pat. Nos. 4,945,050 and 6,194,389.

The nucleic acid can also be introduced into the body in a virus modified to serve as a vehicle without causing pathogenicity. The virus can be, for example, adenovirus, fowlpox virus or vaccinia virus.

miRNAs and siRNAs differ in several ways: miRNA derive from points in the genome different from previously recognized genes, while siRNAs derive from mRNA, viruses or transposons, miRNA derives from hairpin structures, while siRNA derives from longer duplexed RNA, miRNA is conserved among related organisms, while siRNA usually is not, and miRNA silences loci other than that from which it derives, while siRNA silences the loci from which it arises. Interestingly, miRNAs tend not to exhibit perfect complementarity to the mRNA whose expression they inhibit. See, McManus et al., supra. See also, Cheng et al., Nucleic Acids Res. 33(4):1290-7 (2005); Robins and Padgett, Proc Natl Acad Sci USA. 102(11):4006-9 (2005); Brennecke et al., PLoS Biol. 3(3):e85 (2005). Methods of designing miRNAs are known. See, e.g., Zeng et al., Methods Enzymol. 392:371-80 (2005); Krol et al., J Biol. Chem. 279(40):42230-9 (2004); Ying and Lin, Biochem Biophys Res Commun. 326(3):515-20 (2005).

8. Co-Administration with an Inhibitor of Cyclo-Oxygenase

In some embodiments, the invention provides methods of reducing, inhibiting, and/or preventing niacin-induced flushing in an individual in need thereof, by co-administration of (i) an inhibitor of sEH, (ii) an epoxygenated fatty acid, or (iii) both an inhibitor of sEH and an epoxygenated fatty acid, and an inhibitor of cyclo-oxygenase (COX). The inhibitor of cyclo-oxygenase can selectively inhibit COX-2 or inhibit both COX-1 and COX-2. It has been discovered that concurrent inhibition of sEH and cyclo-oxygenase operate cooperatively to reduce inflammation, including prostaglandin mediators of inflammation. See, e.g., WO 2006/086108. Data in the present application show an effect of celecoxib (a COX-2 inhibitor) in counteracting flushing. Dunn et al, Am J. Ther. (1995) 2(7):478-480 showed that 325 mg of aspirin was more effective than 165 mg of aspirin or 200 mg ibuprofen.

Co-administration of (i) an inhibitor of sEH, (ii) an epoxygenated fatty acid, or (iii) both an inhibitor of sEH and an epoxygenated fatty acid and an inhibitor of cyclo-oxygenase can improve the safety and reduce undesirable side effects of the inhibitor of cyclo-oxygenase by allowing the inhibitor of cyclo-oxygenase to be administered at a dose that is subtherapeutic or non-therapeutic to reduce, inhibit or prevent niacin-induced flushing, while still achieving an efficacious response. For example, when co-administered with an epoxygenated fatty acid, an inhibitor of sEH, or mixtures thereof, aspirin can be administered at a dose that is less than about 325 mg, for example, less than about 300 mg, 250 mg, 200 mg, 150 mg, 100 mg or less, and niacin-induced flushing can be effectively reduced, inhibited or prevented. Likewise, when co-administered with an epoxygenated fatty acid, an inhibitor of sEH, or mixtures thereof, ibuprofen can be administered at a dose that is less than about 200 mg, for example, less than about 150 mg, 100 mg, 50 mg, 25 mg, or less, and niacin-induced flushing can be effectively reduced, inhibited or prevented.

All current non-steroidal anti-inflammatory drugs (NSAIDs) inhibit both isoforms, but most tend to inhibit the two isoforms to different degrees. Since COX-2 is considered the enzyme associated with an inflammatory response, enzyme selectivity is generally measured in terms of specificity for COX-2. Typically, cells of a target organ that express COX-1 or COX-2 are exposed to increasing levels of NSAIDs. If the cell does not normally produce COX-2, COX-2 is induced by a stimulant, usually bacterial lipopolysaccharide (LPS).

The relative activity of NSAIDs on COX-1 and COX-2 is expressed by the ratio of IC₅₀s for each enzyme: COX-2 (IC₅₀)/COX-1 (IC₅₀). The smaller the ratio, the more specific the NSAID is for COX-2. For example, various NSAIDs have been reported to have ratios of COX-2 (IC₅₀)/COX-1 (IC₅₀) ranging from 0.33 to 122. See, Englehart et al., J Inflammatory Res 44:422-33 (1995). Aspirin has an IC₅₀ ratio of 0.32, indicating that it inhibits COX-1 more than COX-2, while indomethacin is considered a COX-2 inhibitor since its COX-2 (IC₅₀)/COX-1 (IC₅₀) ratio is 33. Even selective COX-2 inhibitors retain some COX-1 inhibition at therapeutic levels obtained in vivo. Cryer and Feldman, Am J. Med. 104(5):413-21 (1998).

Commercially available NSAIDs which can be used in the methods and compositions of the invention include the traditional NSAIDs diclofenac potassium, diclofenac sodium, diclofenac sodium with misoprostol, diflunisal, etodolac, fenoprofen calcium, flurbiprofen, ibuprofen, indomethacin, ketoprofen, meclofenamate sodium, mefenamic acid, meloxicam, nabumetone, naproxen sodium, piroxicam, tolmetin sodium, the COX-2 inhibitors celecoxib, rofecoxib, and valdecoxib, the acetylated salicylates, such as aspirin, and the non-acetylated salicylates, such as magnesium salicylate, choline salicylate, salsalate, and sodium salicylate.

### 9. Formulation and Therapeutic Administration

Epoxygenated fatty acids and inhibitors of sEH can be prepared and administered in a wide variety of oral, parenteral and aerosol formulations. In some preferred forms, compounds for use in the methods of the present invention can be administered by injection, that is, intravenously, intramuscularly, intracutaneously, subcutaneously, intradermally, topically, intraduodenally, or intraperitoneally, while in others, they are administered orally. Administration can be systemic or local, as desired. The sEH inhibitor or epoxygenated fatty acids, or both, can also be administered by inhalation. Additionally, the sEH inhibitors, or epoxygenated fatty acids, or both, can be administered transdermally. Accordingly, the methods of the invention permit administration of pharmaceutical compositions comprising a pharmaceutically acceptable carrier or excipient and either a selected inhibitor or a pharmaceutically acceptable salt of the inhibitor.

For preparing pharmaceutical compositions from sEH inhibitors, or epoxygenated fatty acids, or both, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

A variety of solid, semisolid and liquid vehicles have been known in the art for years for topical application of agents to the skin. Such vehicles include creams, lotions, gels, balms, oils, ointments and sprays. See, e.g., Provost C. “Transparent oil-water gels: a review,” Int J Cosmet Sci. 8:233-247 (1986), Katz and Poulsen, Concepts in biochemical pharmacology, part I. In: Brodie B B, Gilette J R, eds. Handbook of Experimental Pharmacology. Vol. 28. New York, N.Y.: Springer; 107-174 (1971), and Hadgcraft, “Recent progress in the formulation of vehicles for topical applications,” Br J Dermatol., 81:386-389 (1972). A number of topical formulations of analgesics, including capsaicin (e.g., Capsin®), so-called “counter-irritants” (e.g., Icy-Hot®, substances such as menthol, oil of wintergreen, camphor, or eucalyptus oil compounds which, when applied to skin over an area presumably alter or off-set pain in joints or muscles served by the same nerves) and salicylates (e.g. BenGay®), are known and can be readily adapted for topical administration of sEHI by replacing or combining the active ingredient or ingredient with an sEHI, epoxygenated fatty acids, or mixtures thereof. It is presumed that the person of skill is familiar with these various vehicles and preparations and they need not be described in detail herein.

Inhibitors of sEHI, or epoxygenated fatty acids, or both, (the “agents”) can be mixed into such modalities (creams, lotions, gels, etc.) for topical administration. In general, the concentration of the agents provides a gradient which drives the agent into the skin. Standard ways of determining flux of drugs into the skin, as well as for modifying agents to speed or slow their delivery into the skin are well known in the art and taught, for example, in Osborne and Amann, eds., Topical Drug Delivery Formulations, Marcel Dekker, 1989. The use of dermal drug delivery agents in particular is taught in, for example, Ghosh et al., eds., Transdermal and Topical Drug Delivery Systems, CRC Press, (Boca Raton, Fla., 1997).

In some embodiments, the agents are in a cream. Typically, the cream comprises one or more hydrophobic lipids, with other agents to improve the “feel” of the cream or to provide other useful characteristics. In one embodiment, for example, a cream of the invention may contain 0.01 mg to 10 mg of sEHI, with or without one or more epoxygenated fatty acids, per gram of cream in a white to off-white, opaque cream base of purified water USP, white petrolatum USP, stearyl alcohol NF, propylene glycol USP, polysorbate 60 NF, cetyl alcohol NF, and benzoic acid USP 0.2% as a preservative. In the studies reported in the Examples, sEHI were mixed into a commercially available cream, Vanicream® (Pharmaceutical Specialties, Inc., Rochester, Minn.) comprising purified water, white petrolatum, cetearyl alcohol and ceteareth-20, sorbitol solution, propylene glycol, simethicone, glyceryl monostearate, polyethylene glycol monostearate, sorbic acid and BHT.

In other embodiments, the agent or agents are in a lotion. Typical lotions comprise, for example, water, mineral oil, petrolatum, sorbitol solution, stearic acid, lanolin, lanolin alcohol, cetyl alcohol, glyceryl stearate/PEG-100 stearate, triethanolamine, dimethicone, propylene glycol, microcrystalline wax, tri(PPG-3 myristyl ether) citrate, disodium EDTA, methylparaben, ethylparaben, propylparaben, xanthan gum, butylparaben, and methyldibromo glutaronitrile.

In some embodiments, the agent is, or agents are, in an oil, such as jojoba oil. In some embodiments, the agent is, or agents are, in an ointment, which may, for example, white petrolatum, hydrophilic petrolatum, anhydrous lanolin, hydrous lanolin, or polyethylene glycol. In some embodiments, the agent is, or agents are, in a spray, which typically comprise an alcohol and a propellant. If absorption through the skin needs to be enhanced, the spray may optionally contain, for example, isopropyl myristate.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution. Transdermal administration can be performed using suitable carriers. If desired, apparatuses designed to facilitate transdermal delivery can be employed. Suitable carriers and apparatuses are well known in the art, as exemplified by U.S. Pat. Nos. 6,635,274, 6,623,457, 6,562,004, and 6,274,166.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

A therapeutically effective amount of the sEH inhibitor, or epoxygenated fatty acids, or both, is employed in reducing, alleviating, relieving, ameliorating, preventing and/or inhibiting flushing. The dosage of the specific compound for treatment depends on many factors that are well known to those skilled in the art. They include for example, the route of administration and the potency of the particular compound.

Determination of an effective amount is well within the capability of those skilled in the art. Generally, an efficacious or effective amount of a sEH inhibitor or an epoxygenated fatty acid is determined by first administering a low dose or a small amount of either a sEH inhibitor or an epoxygenated fatty acid, and then incrementally increasing the administered dose or dosages, adding a second medication as needed, until a desired effect of is observed in the treated subject with minimal or no toxic side effects. An exemplary dose of an sEHi or epoxygenated fatty acid is from about 0.001 μM/kg to about 100 mg/kg body weight of the mammal. sEH inhibitors with lower IC50 concentrations can be administered in lower doses.

Efficacious doses of niacin are well known in the art. For example oral and oral extended release formulations of niacin in dosages of from 500 mg-1000 mg are available (e.g., Niaspan, Niacor, generic nicotinic acid or niacin). In some embodiments, the niacin is co-formulated with another cholesterol-lowering or hypolipidemic agent, e.g., lovastatin or simvastatin. Generally, nicotinic acid is often administered at doses from about 50 mg to about 8 grams each day, in single or divided daily doses. Lower dosages can be used initially, and dosages increased to further minimize the flushing effect. The dosages of nicotinic acid receptor agonists other than nicotinic acid vary within wide limits. Nicotinic acid receptor agonists that are useful for treating atherosclerosis can be administered in amounts ranging from as low as about 0.01 mg/kg/day to as high as about 100 mg/kg/day, in single or divided doses. A representative dosage is about 0.1 mg/day to about 2 g/day.

EETs are unstable in acidic conditions, and can be converted to DHETs. To avoid conversion of orally administered EETs to DHETs under the acidic conditions present in the stomach, EETs can be administered intravenously, by injection, or by aerosol. EETs intended for oral administration can be encapsulated in a coating that protects the EETs during passage through the stomach. For example, the EETs can be provided with a so-called “enteric” coating, such as those used for some brands of aspirin, or embedded in a formulation. Such enteric coatings and formulations are well known in the art. In some formulations, the EETs, or a combination of the EETs and an sEH inhibitor are embedded in a slow-release formulation to facilitate administration of the agents over time.

In another set of embodiments, an sEH inhibitor, one or more epoxygenated fatty acids, or both an sEH inhibitor and an epoxygenated fatty acid are administered by delivery to the nose or to the lung. Intranasal and pulmonary delivery are considered to be ways drugs can be rapidly introduced into an organism. Devices for delivering drugs intranasally or to the lungs are well known in the art. The devices typically deliver either an aerosol of an therapeutically active agent in a solution, or a dry powder of the agent. To aid in providing reproducible dosages of the agent, dry powder formulations often include substantial amounts of excipients, such as polysaccharides, as bulking agents.

Detailed information about the delivery of therapeutically active agents in the form of aerosols or as powders is available in the art. For example, the Center for Drug Evaluation and Research (“CDER”) of the U.S. Food and Drug Administration provides detailed guidance in a publication entitled: “Guidance for Industry: Nasal Spray and Inhalation Solution, Suspension, and Spray Drug Products—Chemistry, Manufacturing, and Controls Documentation” (Office of Training and Communications, Division of Drug Information, CDER, FDA, July 2002). This guidance is available in written form from CDER, or can be found on the worldwide web at “fda.gov/cder/guidance/4234fnl.htm”. The FDA has also made detailed draft guidance available on dry powder inhalers and metered dose inhalers. See, Metered Dose Inhaler (MDI) and Dry Powder Inhaler (DPI) Drug Products—Chemistry, Manufacturing, and Controls Documentation, 63 Fed. Reg. 64270, (November 1998). A number of inhalers are commercially available, for example, to administer albuterol to asthma patients, and can be used instead in the methods of the present invention to administer the sEH inhibitor, epoxygenated fatty acid, or a combination of the two agents to subjects in need thereof.

In some aspects of the invention, the sEH inhibitor, epoxygenated fatty acid, or combination thereof, is dissolved or suspended in a suitable solvent, such as water, ethanol, or saline, and administered by nebulization. A nebulizer produces an aerosol of fine particles by breaking a fluid into fine droplets and dispersing them into a flowing stream of gas. Medical nebulizers are designed to convert water or aqueous solutions or colloidal suspensions to aerosols of fine, inhalable droplets that can enter the lungs of a patient during inhalation and deposit on the surface of the respiratory airways. Typical pneumatic (compressed gas) medical nebulizers develop approximately 15 to 30 microliters of aerosol per liter of gas in finely divided droplets with volume or mass median diameters in the respirable range of 2 to 4 micrometers. Predominantly, water or saline solutions are used with low solute concentrations, typically ranging from 1.0 to 5.0 mg/mL.

Nebulizers for delivering an aerosolized solution to the lungs are commercially available from a number of sources, including the AERx™ (Aradigm Corp., Hayward, Calif.) and the Acorn II® (Vital Signs Inc., Totowa, N.J.).

Metered dose inhalers are also known and available. Breath actuated inhalers typically contain a pressurized propellant and provide a metered dose automatically when the patient's inspiratory effort either moves a mechanical lever or the detected flow rises above a preset threshold, as detected by a hot wire anemometer. See, for example, U.S. Pat. Nos. 3,187,748; 3,565,070; 3,814,297; 3,826,413; 4,592,348; 4,648,393; 4,803,978; and 4,896,832.

The formulations may also be delivered using a dry powder inhaler (DPI), i.e., an inhaler device that utilizes the patient's inhaled breath as a vehicle to transport the dry powder drug to the lungs. Such devices are described in, for example, U.S. Pat. Nos. 5,458,135; 5,740,794; and 5,785,049. When administered using a device of this type, the powder is contained in a receptacle having a puncturable lid or other access surface, preferably a blister package or cartridge, where the receptacle may contain a single dosage unit or multiple dosage units.

Other dry powder dispersion devices for pulmonary administration of dry powders include those described in Newell, European Patent No. EP 129985; in Hodson, European Patent No. EP 472598, in Cocozza, European Patent No. EP 467172, and in Lloyd, U.S. Pat. Nos. 5,522,385; 4,668,281; 4,667,668; and 4,805,811. Dry powders may also be delivered using a pressurized, metered dose inhaler (MDI) containing a solution or suspension of drug in a pharmaceutically inert liquid propellant, e.g., a chlorofluorocarbon or fluorocarbon, as described in U.S. Pat. Nos. 5,320,094 and 5,672,581.

The agent can be co-administered with a pharmaceutical agent other than an sEHi or an epoxygenated fatty acid used in the art to alleviate the symptoms of niacin-induced flushing, e.g., aspirin or another non-steroidal anti-inflammatory agent, a vasoconstriction agent (e.g., a topical vasoconstriction agent), an prostaglandin D2 receptor antagonist, e.g., laropiprant (MK-0524A), an inhibitor of transient receptor potential (TRP) channels, etc. See, e.g., WO 2008/097535; WO 2006/089309 and WO 2004/103370. TRP channels are reviewed in, e.g., Latorre, et al., Q Rev Biophys. (2009) 42(3):201-46; Venkatachalam K, Annu Rev Biochem. (2007) 76:387-417 and Myers, et al., Neuron (2007) 54:847-50. The co-administered pharmaceutical agent can be administered in a therapeutically effective or therapeutically ineffective (e.g., subtherapeutic or non-therapeutic) amount.

Without further elaboration, it is believed that one skilled in the art can, using the preceding description, practice the present invention to its fullest extent.

### EXAMPLES

The following examples are offered to illustrate, but not to limit the claimed invention.

### Example 1

### Materials and Methods

Mouse Model: C57BL/6 mice were obtained from Charles River Laboratories (location). sEHi knockout mice were generated at UC Davis using the C57BL/6 background [EnayetAllah, et al., J Biol. Chem. (2008) 283(52):36592-8]. For the experiments, mice were anesthetized using Nembutal (50 mg/kg) given by intraperitoneal (I.P.) injection. Niacin was administered intraperitoneally at a concentration of 30 mg/kg in physiologic saline (equivalent to a human dose of ˜2 grams). sEH inhibitors and other compounds (e.g. aspirin, COX-2 inhibitors) were administered intraperitoneally over a range of relevant doses 30 minutes before niacin.

Laser Doppler ear blood flow: The change in ear flow was measured using a laser doppler flowmeter (BLF 21, on loan from Transonic Systems, Inc., Ithaca, N.Y.). As described by Cheng et al [Proc Natl Acad Sci (2006) 103(17):6682-7] the flow probe was placed against the ventral aspect of the right ear of the anesthetized mouse. The laser doppler probe was fitted with a sleeve of 2 mm length plastic tubing and attached to a micromanipulator to standardize the depth of the tissue being measured. Blood flow was measured at 30 second intervals before and during exposure to compounds. Baseline blood flow was established by the average of measurements over 5 min prior to injection of drug or vehicle. Data were analyzed as a fraction of baseline.

Inhibitors of soluble epoxide hydrolase: To test the concept that inhibition of flushing was a class effect not specific to one compound, experiments were done using the sEH inhibitors TPAU (1-trifluoromethoxyphenyl-3-(1-acetylpiperidin-4-yl) urea, 0.01-1 mg/kg), t-AUCB (4 mg/kg) and sorafenib (4 mg/kg. For comparison, identical experiments were performed using aspirin (4 mg/kg) and celecoxib (4 mg/kg) to inhibit COX-1 and COX-2 pathways. All compounds were administered intraperitoneally 30 minutes before niacin.

Eicosanoid analysis: An LC-MS/MS-based method was used to quantify eicosanoids both from tissue and plasma [Lundstrom, et al., Methods Mol. Biol. (2009) 579:161-87]. Samples were spiked with a suite of odd chain length analogs (surrogates) then solvent extracted and partially purified by passing through a solid phase (SP) extraction column using Oasis HLB cartridges. The loaded column was washed with 2 mL 2.5 mM H2PO4+10% methanol and dried under vacuum. Target analytes were eluted with 2 mL of ethyl acetate. The collected extracts were evaporated to dryness under nitrogen, re-dissolved in a final volume of 100 μL with methanol containing deuterated isomers of internal standards including prostaglandins, thromboxanes, epoxides, diols, of arachidonic acid, which allows accurate surrogate recovery calculations and stored at −20° C. until analysis. A 10-20 μL aliquot of each sample was separated by reversed phase HPLC chromatography on a 150 mm×2 mm I.D. 5 μm particle size C18 column using a gradient of water-0.1% acetic acid and 89:11 acetonitrile/methanol (v/v)-0.1% acetic acid. The separated analytes were quantified using negative mode electrospray ionization and tandem mass spectrometry in multi-reaction monitoring mode (MRM, Waters Alliance 2795 LC system and Quattro Ultima tandem-quadrupole mass spectrometer, Micromass). The system was calibrated with a minimum of five calibration solutions containing analytical targets at concentrations ranging from 1 to 1000 nM. The analysis of each sample was repeated three times. Calibration check solutions were analyzed at a minimum frequency of 10 hours to ensure stability of the analytical calibration throughout a given analysis.

### Example 2

### Inhibitors of Soluble Epoxide Hydrolase (sEH) do not Reduce Levels of Prostaglandin D2 (PGD2) in the Presence of Niacin

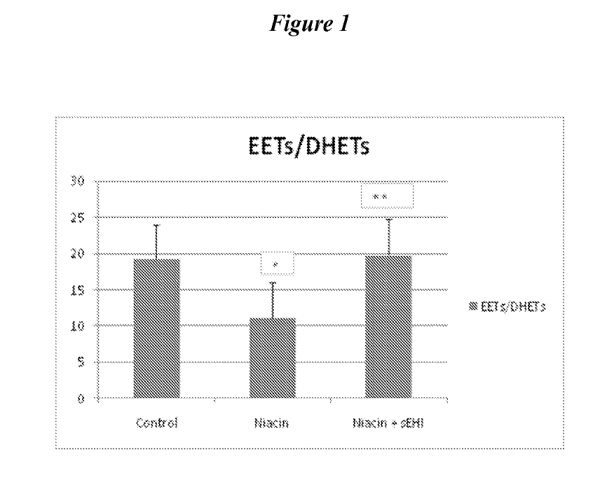

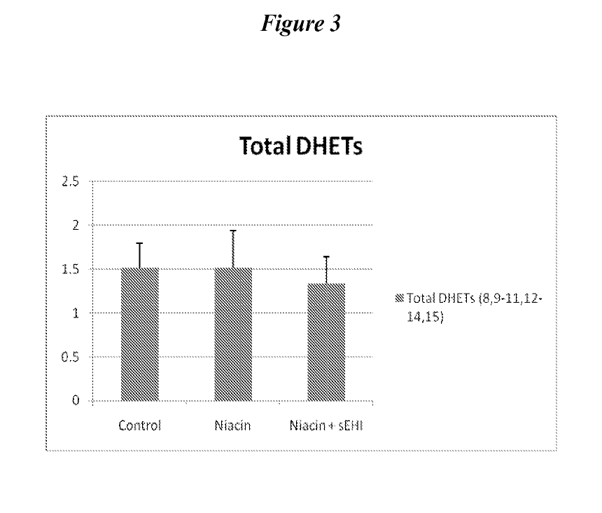

Subcutaneous administration of niacin at a dose of 30 mg/kg alone (i.e., without sEHi) reduced plasma levels of EETs, either as total EETs or as the ratio of EETs/DHETs (FIG. 1-3). These data suggest that niacin, at least acutely, decreases the favorable endogenous balance of vasodilatory vs vasoconstrictive prostaglandins. Treatment with sEHi (TPAU, 3 mg/kg) counters this reduction in EETs, returning the physiologic balance of EETs to DHETs.

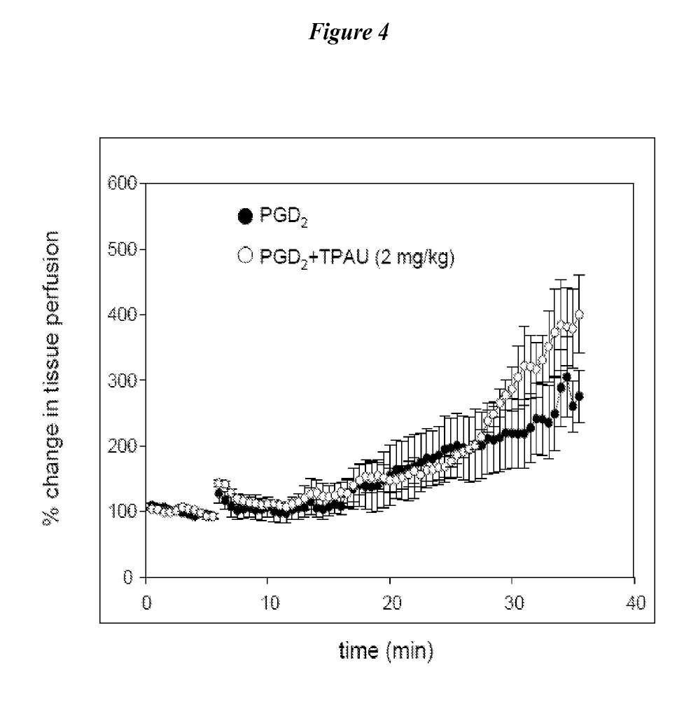

Since prior studies (Cheng, et al., (2006) Proc Natl Acad Sci 103(17):6682-7) showed that the flushing response to niacin was largely due to PGD2 stimulation of the DP1 receptor, we examined whether the sEH inhibitor TPAU could block PDG2 induced vasodilation. Pretreatment with a concentration of TPAU that was clearly sufficient to inhibit the niacin-induced increase in tissue perfusion did not limit the increase in perfusion with administration of PGD2 (FIG. 4). Thus, inhibition of sEH, in this model, did not directly limit PGD2 induced vasodilation.

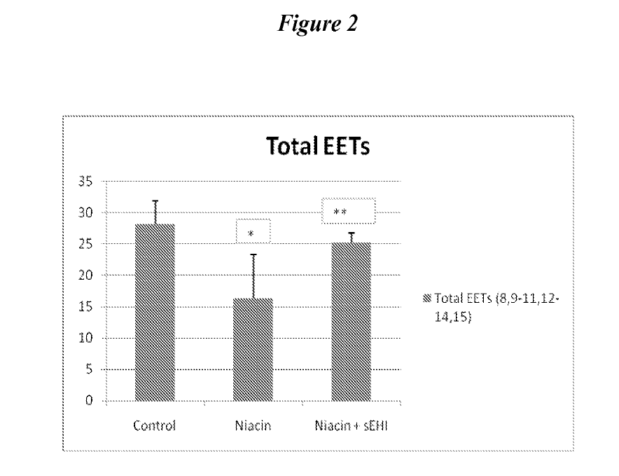

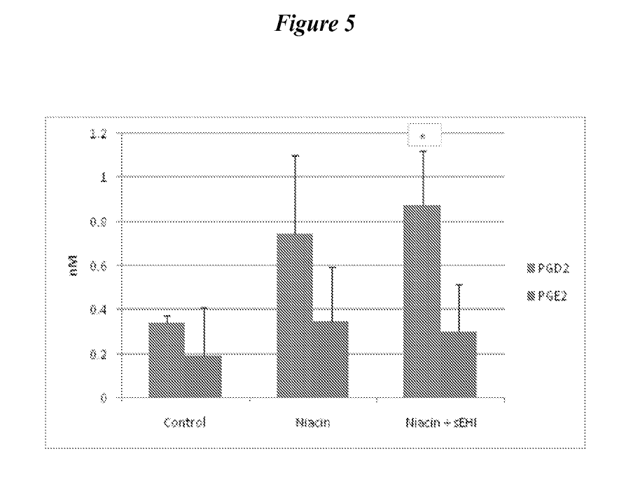

The effect of niacin and sEHi on prostaglandins was further explored by measuring PGD2 and PGE2 in serum of experimental animals exposed to niacin +/−TPAU. Niacin did, as expected, increase PGD2 levels by approximately 100% (FIG. 4.) without altering PGE2 levels. As seen in FIG. 5, PGD2, but not PGE2, increased two-fold following administration of niacin alone. Surprisingly, there was no reduction in PGD2 with niacin+sEHi. When metabolites of the P450 system were measured, it was found that niacin alone significantly reduced the serum levels of EET's and the ratio of EET's/DHET's, while these changes were completely reversed by pre-administration of an sEH inhibitor (FIGS. 1 and 2).

As the flushing effect of niacin is essentially local rather than systemic, the effect of niacin +/−sEHi on ear tissue prostaglandins were also examined by LC-MS. Consistent with the serum data, niacin increased tissue PGD2 by 21% (26045 to 33214, P=0.09, while pre-treatment with an sEHi significantly increased PGD2 to 42087 (P<0.05 vs both control and niacin alone).

### Example 3

### Knocking Out sEH Gene Prevents Niacin-Induced Flushing

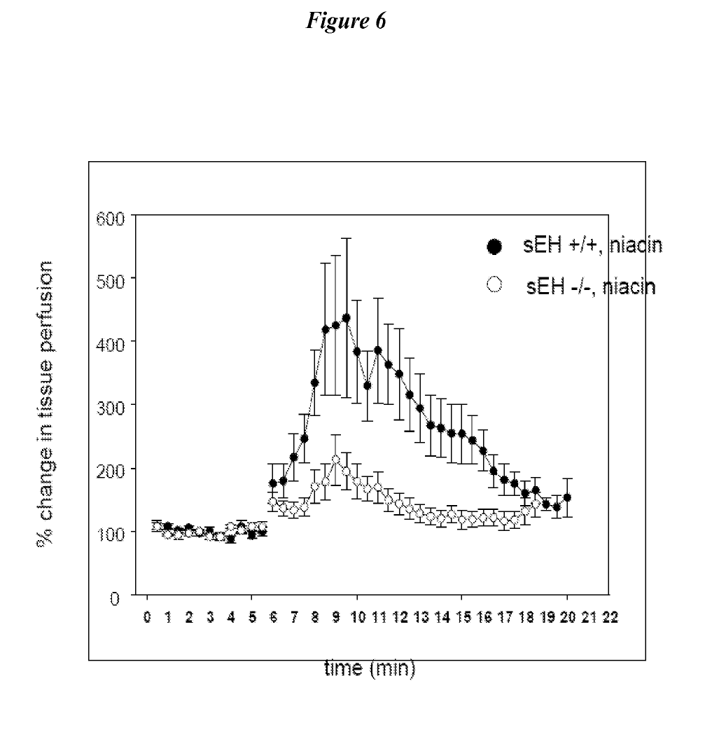

Male C57BL/6 wild type mice (n=6, closed circles) were anesthetized with Nembutal™ (50 mg/kg) and ear cutaneous perfusion was measured for 5 minutes to obtain a baseline. Animals were then injected with a niacin solution (30 mg/kg, in physiological saline) and further monitored for 15 minutes. Niacin in these animals induced a rapid increase in blood flow as shown by an increased perfusion rate. A similar procedure was followed for a group of mice (n=6, open circles) that lack the sEH enzyme. These animals were generated using the C57BL/6 background. In sEH null mice the flushing response to niacin (30 mg/kg) was greatly attenuated. The results are shown in FIG. 6

### Example 4

### Chemical Inhibition of sEH Dose-Dependently Reverses Flushing in Mice

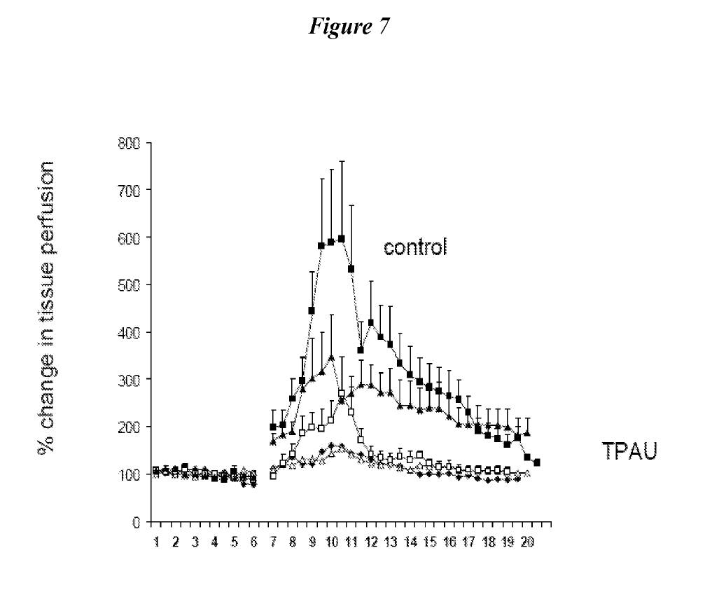

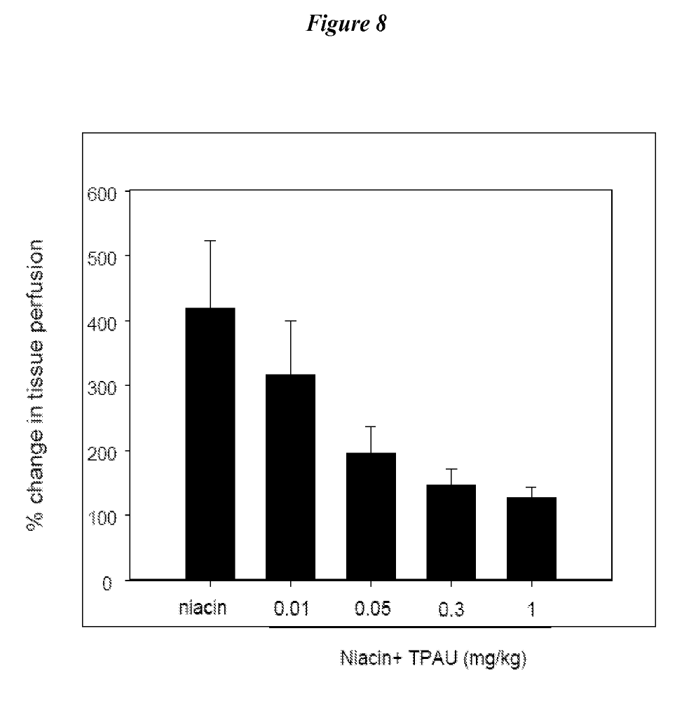

Mice were anesthetized with Nembutal™ (50 mg/kg) and ear cutaneous perfusion was measured as explained above. Animals were then injected with a niacin solution (30 mg/kg, in physiological saline) and response (% tissue perfusion) after 3 minutes measured. A potent inhibitor of sEH (TPAU) dose-dependently reduced flushing when administered 1 hour prior to niacin administration (n=4-6) [p>0.001, Univariate ANOVA, followed by post hoc LSD test]. FIG. 7 shows the dose response after niacin administration, without and with pre-treatment with the sEH inhibitor TPAU (0.01 to 1 mg/kg). As seen in FIG. 8, there was a significant reduction in peak perfusion with TPAU 0.05 mg/kg to 1 mg/kg.

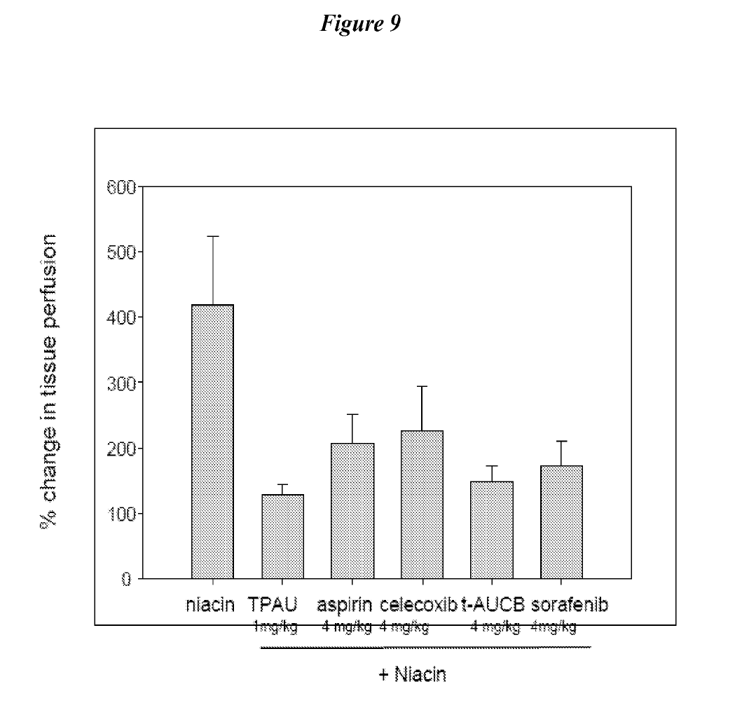

The composite results of experiments using 3 sEH inhibitors (TPAU, t-AUCB, and sorafenib), and well as aspirin and celecoxib are shown in FIG. 9. Each intervention significantly reduced peak ear tissue perfusion, without significant differences between them.

### Example 5

### sEHi Reduces Niacin-Induced Increases in 5-Lipoxygenase Pathway Products

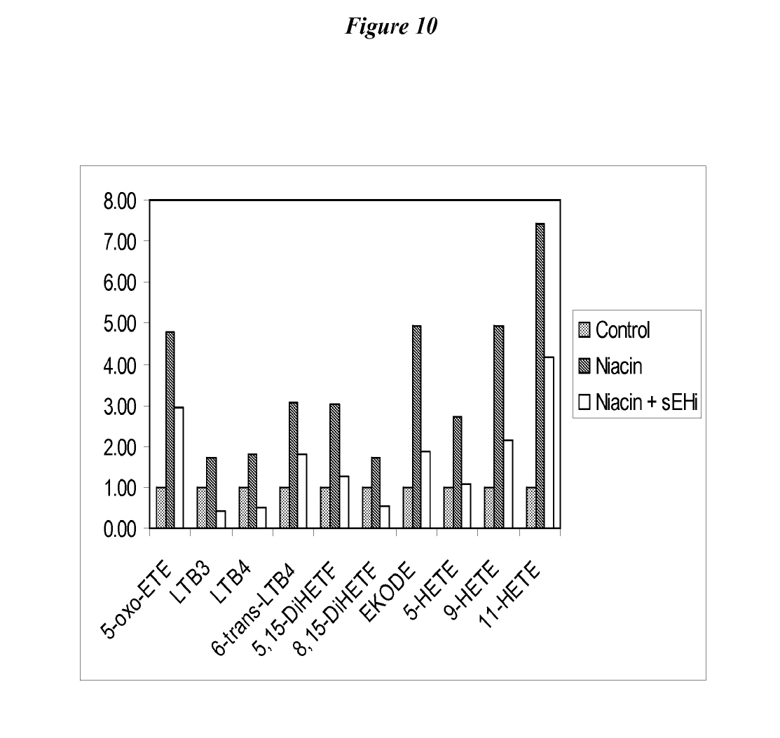

Niacin 30 mg/kg subcutaneously increased products of the 5-lipoxygenase pathway. The murine model of flushing discussed herein is representative of niacin-induced flushing in humans. Therefore, the human lipoxygenase pathway is adversely influenced by niacin. The increased products of the 5-lipoxygenase pathway are depicted in FIG. 10, which shows the relative increases of metabolites in ear tissue samples in mice administered niacin and an sEHi (TPAU, 30 mg/kg) compared to mice administered niacin alone and untreated control mice. As shown in FIG. 10, pre-treatment with the sEH inhibitor TPAU 3 mg/kg limited or abrogated niacin-induced increases in products of the 5-lipoxygenase pathway. Products of the 5-lipoxygenase pathway are generally considered pro-inflammatory. Therefore, these data are consistent with the conclusion that niacin alone results in pro-inflammatory changes in eicosanoids, and that concurrent treatment with an sEH inhibitor can largely prevent these adverse changes.

### Example 6

### Desensitization of Transient Receptor Potential (“TRP”) Channels Blunts the Flushing Response to Niacin

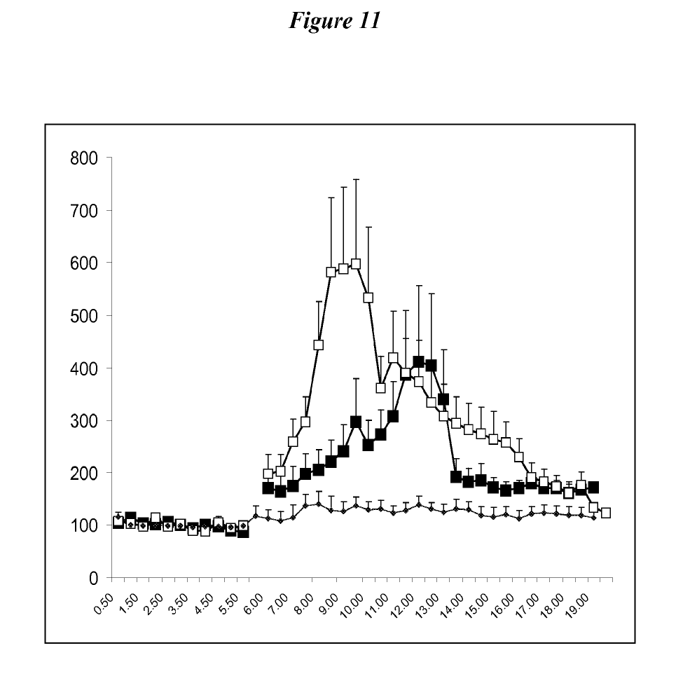

Tachyphylaxis to the flushing effect of niacin is well known, although the mechanism has not been elucidated. As with niacin, capsaicin is well known to cause local vasodilation and result in tachyphylaxis after repeated exposure. Tachyphylaxis to the vasodilatory effect of capsaicin is likely mediated by repetitive activation of transient receptor potential channels, in particular TRPV1, with resultant desensitization and internalization. See, Fan et al., J Biol Chem (2009) 284:27884-91; Myers, et al., Neuron (2007) 54:847-50. When applied to the mouse ear, capsaicin resulted in a time-dependent increase in tissue perfusion similar to that seen with PGD2. Subsequently, mice were exposed to capsaicin daily for 3 days (treated topically, 4 times per day), resulting in abolition of the acute vasodilatory response (i.e. tachyphylaxis to capsaicin). Following tachyphylaxis to capsaicin, acute exposure to niacin resulted in a blunted vasodilatory response, with a reduced initial flushing response, leaving only the secondary peak (FIG. 11). Although the exact sequence of events following niacin exposure is not clear it seems that niacin activates a number of targets simultaneously. These data are consistent with the first phase of niacin-induced flushing involving activation of TRPV1 channels and is also consistent with the hypothesis that tachyphylaxis to niacin flushing is due to desensitization of TRP channels.

### Example 7

### Desensitization of TRP Channels+sEH Inhibition Abolishes the Flushing Response to Niacin

To test whether the partial reduction in niacin-induced flushing from capsaicin tachyphylaxis was related to, or independent of, the reduction in flushing due to sEH inhibition, we used the properties of capsaicin as an sEH inhibitor at moderate concentrations (IC₅₀ of capsaicin on recombinant murine and human sEH is 1.5 μM). After tachyphylaxis was established by repeated exposure to capsaicin, animals were then exposed acutely to topical capsaicin prior to niacin injection. This experiment therefore combined TRP channel desensitization (capsaicin tachyphylaxis) with sEH inhibition (acute capsaicin exposure). In contrast to only chronic exposure to capsaicin, the flushing response to niacin was abolished, with loss of the second peak of vasodilation. Thus, our data indicate that the flushing response to niacin is a multi-component complex biological event that is not driven solely by PGD2 as often proposed in the literature. Rather, the involvement of other biological pathways in niacin induced flushing is important and provides alternate strategies of limiting flushing.

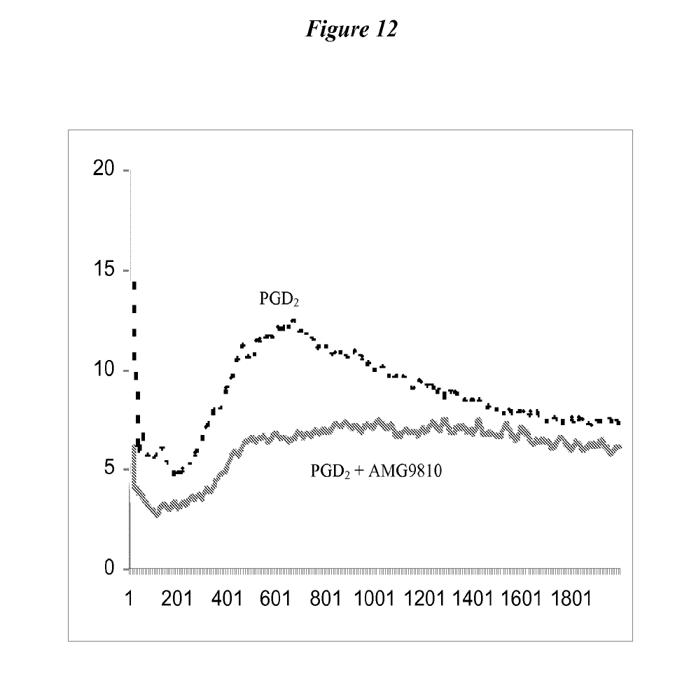

The role of TRP channels was further supported by experiments in which the highly potent and selective TRP channel inhibitor, AMG9810 (10 mg/kg IP) limited the flushing response to both niacin and PGD2 (Gavva, et al., J Pharmacol Exp Ther (2005) 313:474-84). As seen in FIG. 12, AMG9810 blunted the biologic response to PGD2. Importantly, AMG9810 abolished the flushing response to niacin in the animal tested. This may have been due to blockade of TRP channels activated by both PGD2 and LOX metabolites.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Informal Sequence Listing

| SEQ ID NO: 1 | |
|---|---|
| 1 | ggcacgagct ctctctctct ctctctctct ctctcgccgc catgacgctg cgcggcgccg |
| 61 | tcttcgacct tgacggggtg ctggcgctgc cagcggtgtt cggcgtcctc ggccgcacgg |
| 121 | aggaggccct ggcgctgccc agaggacttc tgaatgatgc tttccagaaa gggggaccag |
| 181 | agggtgccac tacccggctt atgaaaggag agatcacact ttcccagtgg ataccactca |
| 241 | tggaagaaaa ctgcaggaag tgctccgaga ccgctaaagt ctgcctcccc aagaatttct |
| 301 | ccataaaaga aatctttgac aaggcgattt cagccagaaa gatcaaccgc cccatgctcc |
| 361 | aggcagctct catgctcagg aagaaaggat tcactactgc catcctcacc aacacctggc |
| 421 | tggacgaccg tgctgagaga gatggcctgg cccagctgat gtgtgagctg aagatgcact |
| 481 | ttgacttcct gatagagtcg tgtcaggtgg gaatggtcaa acctgaacct cagatctaca |
| 541 | agtttctgct ggacaccctg aaggccagcc ccagtgaggt cgtttttttg gatgacatcg |
| 601 | gggctaatct gaagccagcc cgtgacttgg gaatggtcac catcctggtc caggacactg |
| 661 | acacggccct gaaagaactg gagaaagtga ccggaatcca gcttctcaat accccggccc |
| 721 | ctctgccgac ctcttgcaat ccaagtgaca tgagccatgg gtacgtgaca gtaaagccca |
| 781 | gggtccgtct gcattttgtg gagctgggct ggcctgctgt gtgcctctgc catggatttc |
| 841 | ccgagagttg gtattcttgg aggtaccaga tccctgctct ggcccaggca ggttaccggg |
| 901 | tcctagctat ggacatgaaa ggctatggag agtcatctgc tcctcccgaa atagaagaat |
| 961 | attgcatgga agtgttatgt aaggagatgg taaccttcct ggataaactg ggcctctctc |
| 1021 | aagcagtgtt cattggccat gactggggtg gcatgctggt gtggtacatg gctctcttct |
| 1081 | accccgagag agtgagggcg gtggccagtt tgaatactcc cttcatacca gcaaatccca |
| 1141 | acatgtcccc tttggagagt atcaaagcca acccagtatt tgattaccag ctctacttcc |
| 1201 | aagaaccagg agtggctgag gctgaactgg aacagaacct gagtcggact ttcaaaagcc |
| 1261 | tcttcagagc aagcgatgag agtgttttat ccatgcataa agtctgtgaa gcgggaggac |
| 1321 | tttttgtaaa tagcccagaa gagcccagcc tcagcaggat ggtcactgag gaggaaatcc |
| 1381 | agttctatgt gcagcagttc aagaagtctg gtttcagagg tcctctaaac tggtaccgaa |
| 1441 | acatggaaag gaactggaag tgggcttgca aaagcttggg acggaagatc ctgattccgg |
| 1501 | ccctgatggt cacggcggag aaggacttcg tgctcgttcc tcagatgtcc cagcacatgg |
| 1561 | aggactggat tccccacctg aaaaggggac acattgagga ctgtgggcac tggacacaga |
| 1621 | tggacaagcc aaccgaggtg aatcagatcc tcattaagtg gctggattct gatgcccgga |
| 1681 | acccaccggt ggtctcaaag atgtagaacg cagcgtagtg cccacgctca gcaggtgtgc |
| 1741 | catccttcca cctgctgggg caccattctt agtatacaga ggtggcctta cacacatctt |
| 1801 | gcatggatgg cagcattgtt ctgaaggggt ttgcagaaaa aaaagatttt ctttacataa |
| 1861 | agtgaatcaa atttgacatt attttagatc ccagagaaat caggtgtgat tagttctcca |
| 1921 | ggcatgaatg catcgtccct ttatctgtaa gaacccttag tgtcctgtag ggggacagaa |
| 1981 | tggggtggcc aggtggtgat ttctctttga ccaatgcata gtttggcaga aaaatcagcc |
| 2041 | gttcatttag aagaatctta gcagagattg ggatgcctta ctcaataaag ctaagatgac |

SEQ ID NO: 2
MTLRGAVFDLDGVLALPAVFGVLGRTEEALALPRGLLNDAFQKGGPEGATTRLMKGEITLSQWIPLMEENCRKCSET
AKVCLPKNFSIKEIFDKAISARKINRPMLQAALMLRKKGFTTAILTNTWLDDRAERDGLAQLMCELKMHFDFLIESC
QVGMVKPEPQIYKFLLDTLKASPSEVVFLDDIGANLKPARDLGMVTILVQDTDTALKELEKVTGIQLLNTPAPLPTS
CNPSDMSHGYVTVKPRVRLHFVELGWPAVCLCHGFPESWYSWRYQIPALAQAGYRVLAMDMKGYGESSAPPEIEEYC
MEVLCKEMVTFLDKLGLSQAVFIGHDWGGMLVWYMALFYPERVRAVASLNTPFIPANPNMSPLESIKANPVFDYQLY
FQEPGVAEAELEQNLSRTFKSLFRASDESVLSMHKVCEAGGLFVNSPEEPSLSRMVTEEEIQFYVQQFKKSGFRGPL
NWYRNMERNWKWACKSLGRKILIPALMVTAEKDFVLVPQMSQHMEDWIPHLKRGHIEDCGHWTQMDKPTEVNQILIK
WLDSDARNPPVVSKM

What is claimed is:

1. A method of reducing or preventing niacin-induced cutaneous vasodilation in a subject in need thereof, said method comprising administering to said subject an effective amount of an agent or agents selected from the group consisting of (i) an inhibitor of sEH, (ii) an epoxygenated fatty acid, or (iii) both an inhibitor of sEH and an epoxygenated fatty acid, thereby reducing or preventing said niacin-induced cutaneous vasodilation in said subject.

2. A method of claim 1, wherein the epoxygenated fatty acid is an EET.

3. A method of claim 2, wherein the EET is selected from the group consisting of 14,15-EET, 8,9-EET, 11,12-EET or 5,6-EET.

4. A method of claim 2, wherein the EET is synthetic or an EET analog.

5. A method of claim 1, wherein the agent is an inhibitor of sEH.

6. A method of claim 1, wherein the epoxygenated fatty acid is an epoxide of linoleic acid, eicosapentaenoic acid (“EPA”) or docosahexaenoic acid (“DHA”), or a mixture thereof.

7. A method of claim 5, wherein the inhibitor of sEH has a primary pharmacophore selected from the group consisting of a urea, a carbamate and an amide.

8. A method of claim 1, wherein the agent is administered systemically.

9. A method of claim 8, wherein the agent is administered orally.

10. A method of claim 1, wherein the agent is administered locally.

11. A method of claim 10, wherein the agent is administered topically.

12. A method of claim 1, wherein the subject is a human.

13. A method of claim 1, wherein the subject has a blood concentration of niacin of at least 0.1 mM.

14. A method of claim 1, wherein the subject is experiencing cutaneous vasodilation.

15. A method of claim 1, wherein the agent is co-administered with a therapeutically effective amount of niacin.

16. A method of claim 1, wherein the agent is co-administered with a therapeutically effective amount of aspirin.

17. A method of claim 1, wherein the agent is co-administered with a therapeutically effective amount of laropiprant.


SEQUENCE LISTING (38 sequences)

SEQ ID NO: 1 - DNA, 2100 bp, Homo sapiens
  soluble epoxide hydrolase (sEH), cytosolic epoxide hydrolase
ggcacgagctctctctctctctctctctctctctcgccgccatgacgctgcgcggcgccgtcttcgaccttgacggggtgctggcgctgccagcggtgttcggcgtcctcggccgcacggaggaggccctggcgctgcccagaggacttctgaatgatgctttccagaaagggggaccagagggtgccactacccggcttatgaaaggagagatcacactttcccagtggataccactcatggaagaaaactgcaggaagtgctccgagaccgctaaagtctgcctccccaagaatttctccataaaagaaatctttgacaaggcgatttcagccagaaagatcaaccgccccatgctccaggcagctctcatgctcaggaagaaaggattcactactgccatcctcaccaacacctggctggacgaccgtgctgagagagatggcctggcccagctgatgtgtgagctgaagatgcactttgacttcctgatagagtcgtgtcaggtgggaatggtcaaacctgaacctcagatctacaagtttctgctggacaccctgaaggccagccccagtgaggtcgtttttttggatgacatcggggctaatctgaagccagcccgtgacttgggaatggtcaccatcctggtccaggacactgacacggccctgaaagaactggagaaagtgaccggaatccagcttctcaataccccggcccctctgccgacctcttgcaatccaagtgacatgagccatgggtacgtgacagtaaagcccagggtccgtctgcattttgtggagctgggctggcctgctgtgtgcctctgccatggatttcccgagagttggtattcttggaggtaccagatccctgctctggcccaggcaggttaccgggtcctagctatggacatgaaaggctatggagagtcatctgctcctcccgaaatagaagaatattgcatggaagtgttatgtaaggagatggtaaccttcctggataaactgggcctctctcaagcagtgttcattggccatgactggggtggcatgctggtgtggtacatggctctcttctaccccgagagagtgagggcggtggccagtttgaatactcccttcataccagcaaatcccaacatgtcccctttggagagtatcaaagccaacccagtatttgattaccagctctacttccaagaaccaggagtggctgaggctgaactggaacagaacctgagtcggactttcaaaagcctcttcagagcaagcgatgagagtgttttatccatgcataaagtctgtgaagcgggaggactttttgtaaatagcccagaagagcccagcctcagcaggatggtcactgaggaggaaatccagttctatgtgcagcagttcaagaagtctggtttcagaggtcctctaaactggtaccgaaacatggaaaggaactggaagtgggcttgcaaaagcttgggacggaagatcctgattccggccctgatggtcacggcggagaaggacttcgtgctcgttcctcagatgtcccagcacatggaggactggattccccacctgaaaaggggacacattgaggactgtgggcactggacacagatggacaagccaaccgaggtgaatcagatcctcattaagtggctggattctgatgcccggaacccaccggtggtctcaaagatgtagaacgcagcgtagtgcccacgctcagcaggtgtgccatccttccacctgctggggcaccattcttagtatacagaggtggccttacacacatcttgcatggatggcagcattgttctgaaggggtttgcagaaaaaaaagattttctttacataaagtgaatcaaatttgacattattttagatcccagagaaatcaggtgtgattagttctccaggcatgaatgcatcgtccctttatctgtaagaacccttagtgtcctgtagggggacagaatggggtggccaggtggtgatttctctttgaccaatgcatagtttggcagaaaaatcagccgttcatttagaagaatcttagcagagattgggatgccttactcaataaagctaagatgac

SEQ ID NO: 2 - protein, 554 aa, Homo sapiens
  soluble epoxide hydrolase (sEH), cytosolic epoxide hydrolase
MTLRGAVFDLDGVLALPAVFGVLGRTEEALALPRGLLNDAFQKGGPEGATTRLMKGEITLSQWIPLMEENCRKCSETAKVCLPKNFSIKEIFDKAISARKINRPMLQAALMLRKKGFTTAILTNTWLDDRAERDGLAQLMCELKMHFDFLIESCQVGMVKPEPQIYKFLLDTLKASPSEVVFLDDIGANLKPARDLGMVTILVQDTDTALKELEKVTGIQLLNTPAPLPTSCNPSDMSHGYVTVKPRVRLHFVELGWPAVCLCHGFPESWYSWRYQIPALAQAGYRVLAMDMKGYGESSAPPEIEEYCMEVLCKEMVTFLDKLGLSQAVFIGHDWGGMLVWYMALFYPERVRAVASLNTPFIPANPNMSPLESIKANPVFDYQLYFQEPGVAEAELEQNLSRTFKSLFRASDESVLSMHKVCEAGGLFVNSPEEPSLSRMVTEEEIQFYVQQFKKSGFRGPLNWYRNMERNWKWACKSLGRKILIPALMVTAEKDFVLVPQMSQHMEDWIPHLKRGHIEDCGHWTQMDKPTEVNQILIKWLDSDARNPPVVSKM

SEQ ID NO: 3 - DNA, 23 bp, Artificial Sequence
  synthetic soluble epoxide hydrolase (sEH) target sequence
cagtgttcattggccatgactgg

SEQ ID NO: 4 - DNA, 21 bp, Artificial Sequence
  synthetic soluble epoxide hydrolase (sEH) sense-siRNA
guguucauuggccaugacutt

SEQ ID NO: 5 - DNA, 21 bp, Artificial Sequence
  synthetic soluble epoxide hydrolase (sEH) antisense-siRNA
agucauggccaaugaacactt

SEQ ID NO: 6 - DNA, 23 bp, Artificial Sequence
  synthetic soluble epoxide hydrolase (sEH) target sequence
gaaaggctatggagagtcatctg

SEQ ID NO: 7 - DNA, 21 bp, Artificial Sequence
  synthetic soluble epoxide hydrolase (sEH) sense-siRNA
aaggcuauggagagucauctt

SEQ ID NO: 8 - DNA, 21 bp, Artificial Sequence
  synthetic soluble epoxide hydrolase (sEH) antisense-siRNA
gaugacucuccauagccuutt

SEQ ID NO: 9 - DNA, 23 bp, Artificial Sequence
  synthetic soluble epoxide hydrolase (sEH) target sequence
aaaggctatggagagtcatctgc

SEQ ID NO: 10 - DNA, 21 bp, Artificial Sequence
  synthetic soluble epoxide hydrolase (sEH) sense-siRNA
aggcuauggagagucaucutt

SEQ ID NO: 11 - DNA, 21 bp, Artificial Sequence
  synthetic soluble epoxide hydrolase (sEH) antisense-siRNA
agaugacucuccauagccutt

SEQ ID NO: 12 - DNA, 23 bp, Artificial Sequence
  synthetic soluble epoxide hydrolase (sEH) target sequence
caagcagtgttcattggccatga

SEQ ID NO: 13 - DNA, 21 bp, Artificial Sequence
  synthetic soluble epoxide hydrolase (sEH) sense-siRNA
agcaguguucauuggccautt

SEQ ID NO: 14 - DNA, 21 bp, Artificial Sequence
  synthetic soluble epoxide hydrolase (sEH) antisense-siRNA
auggccaaugaacacugcutt

SEQ ID NO: 15 - DNA, 23 bp, Artificial Sequence
  synthetic soluble epoxide hydrolase (sEH) target sequence
cagcacatggaggactggattcc

SEQ ID NO: 16 - DNA, 21 bp, Artificial Sequence
  synthetic soluble epoxide hydrolase (sEH) sense-siRNA
gcacauggaggacuggauutt

SEQ ID NO: 17 - DNA, 21 bp, Artificial Sequence
  synthetic soluble epoxide hydrolase (sEH) antisense-siRNA
aauccaguccuccaugugctt

SEQ ID NO: 18 - DNA, 9 bp, Artificial Sequence
  synthetic linker section short spacer
ttcaagaga

SEQ ID NO: 19 - DNA, 23 bp, Artificial Sequence
  synthetic soluble epoxide hydrolase (sEH) target sequence
cagtgttcattggccatgactgg

SEQ ID NO: 20 - DNA, 59 bp, Artificial Sequence
  synthetic soluble epoxide hydrolase (sEH) hairpin siRNA sense strand
gatccccgtgttcattggccatgactttcaagagaagtcatggccaatgaacacttttt

SEQ ID NO: 21 - DNA, 59 bp, Artificial Sequence
  synthetic soluble epoxide hydrolase (sEH) hairpin siRNA antisense strand
agctaaaaagtgttcattggccatgacttctcttgaaagtcatggccaatgaacacggg

SEQ ID NO: 22 - DNA, 23 bp, Artificial Sequence
  synthetic soluble epoxide hydrolase (sEH) target sequence
gaaaggctatggagagtcatctg

SEQ ID NO: 23 - DNA, 59 bp, Artificial Sequence
  synthetic soluble epoxide hydrolase (sEH) hairpin siRNA sense strand
gatccccaaggctatggagagtcatcttcaagagagatgactctccatagccttttttt

SEQ ID NO: 24 - DNA, 59 bp, Artificial Sequence
  synthetic soluble epoxide hydrolase (sEH) hairpin siRNA antisense strand
agctaaaaaaaggctatggagagtcatctctcttgaagatgactctccatagccttggg

SEQ ID NO: 25 - DNA, 23 bp, Artificial Sequence
  synthetic soluble epoxide hydrolase (sEH) target sequence
aaaggctatggagagtcatctgc

SEQ ID NO: 26 - DNA, 59 bp, Artificial Sequence
  synthetic soluble epoxide hydrolase (sEH) hairpin siRNA sense strand
gatccccaggctatggagagtcatctttcaagagaagatgactctccatagcctttttt

SEQ ID NO: 27 - DNA, 59 bp, Artificial Sequence
  synthetic soluble epoxide hydrolase (sEH) hairpin siRNA antisense strand
agctaaaaaaggctatggagagtcatcatctcttgaaagatgactctccatagcctggg

SEQ ID NO: 28 - DNA, 23 bp, Artificial Sequence
  synthetic soluble epoxide hydrolase (sEH) target sequence
caagcagtgttcattggccatga

SEQ ID NO: 29 - DNA, 59 bp, Artificial Sequence
  synthetic soluble epoxide hydrolase (sEH) hairpin siRNA sense strand
gatccccagcagtgttcattggccatttcaagagaatggccaatgaacactgctttttt

SEQ ID NO: 30 - DNA, 59 bp, Artificial Sequence
  synthetic soluble epoxide hydrolase (sEH) hairpin siRNA antisense strand
agctaaaaaagcagtgttcattggccattctcttgaaatggccaatgaacactgctggg

SEQ ID NO: 31 - DNA, 23 bp, Artificial Sequence
  synthetic soluble epoxide hydrolase (sEH) target sequence
cagcacatggaggactggattcc

SEQ ID NO: 32 - DNA, 59 bp, Artificial Sequence
  synthetic soluble epoxide hydrolase (sEH) hairpin siRNA sense strand
gatccccgcacatggaggactggattttcaagagaaatccagtcctccatgtgcttttt

SEQ ID NO: 33 - DNA, 59 bp, Artificial Sequence
  synthetic soluble epoxide hydrolase (sEH) hairpin siRNA antisense strand
agctaaaaagcacatggaggactggatttctcttgaaaatccagtcctccatgtgcggg

SEQ ID NO: 34 - RNA, 20 nt, Artificial Sequence
  Description of Artificial Sequence Synthetic soluble epoxide hydrolase (sEH) antisense sequence
uguccagugcccacaguccu

SEQ ID NO: 35 - RNA, 20 nt, Artificial Sequence
  Description of Artificial Sequence Synthetic soluble epoxide hydrolase (sEH) antisense sequence
uucccaccugacacgacucu

SEQ ID NO: 36 - RNA, 20 nt, Artificial Sequence
  Description of Artificial Sequence Synthetic soluble epoxide hydrolase (sEH) antisense sequence
guucagccucagccacuccu

SEQ ID NO: 37 - RNA, 19 nt, Artificial Sequence
  Description of Artificial Sequence Synthetic soluble epoxide hydrolase (sEH) antisense sequence
aguccucccgcuucacaga

SEQ ID NO: 38 - RNA, 21 nt, Artificial Sequence
  Description of Artificial Sequence Synthetic soluble epoxide hydrolase (sEH) antisense sequence
gcccacuuccaguuccuuucc